(19) United States
(12) Patent Application Publication
(10) Pub. No.: US 2016/0176796 A1
(43) Pub. Date: Jun. 23, 2016

(54) ACETALDEHYDE PRODUCTION METHOD

(71) Applicant: DAICEL CORPORATION, Osaka-shi, Osaka (JP)

(72) Inventors: Masato KAWABE, Himeji-shi (JP); Yoshihisa MIZUTANI, Himeji-shi (JP)

(73) Assignee: DAICEL CORPORATION, Osaka-shi, Osaka (JP)

(21) Appl. No.: 14/910,208

(22) PCT Filed: Aug. 5, 2014
(86) PCT No.: PCT/JP2014/070597

§ 371 Date: Feb. 4, 2016

(30) Foreign Application Priority Data
Aug. 8, 2013 (JP) 2013-165622
Aug. 19, 2013 (JP) 2013-169907
Aug. 27, 2013 (JP) 2013-175179
Aug. 27, 2013 (JP) 2013-175557
Oct. 28, 2013 (JP) 2013-223356
Apr. 10, 2014 (JP) 2014-081441
Apr. 10, 2014 (JP) 2014-081442
Apr. 10, 2014 (JP) 2014-081443
Apr. 10, 2014 (JP) 2014-081444
Apr. 10, 2014 (JP) 2014-081445

(51) Int. Cl. C07C 45/41; B01D 3/00; C07C 67/08

(52) U.S. Cl. CPC C07C 45/41; C07C 67/08; B01D 3/009

(57) ABSTRACT

Provided is a method for producing high-purity acetaldehyde from acetic acid inexpensively and industrially efficiently.

The present invention relates to a method for producing acetaldehyde via acetic acid hydrogenation. The method hydrogenates acetic acid to give a reaction fluid. The reaction fluid is charged into an absorber. From the reaction fluid, condensed components are absorbed with an absorbing liquid, and non-condensable gases are dissolved in the absorbing liquid. A bottom liquid of the absorber is decompressed (reduced in pressure) to strip the dissolved non-condensable gases from the absorbing liquid. The residual liquid after the non-condensable gas stripping is recycled to the absorber.

### TECHNICAL FIELD

The present invention relates to methods for producing acetaldehyde via hydrogenation of acetic acid. The present invention also relates to methods for producing acetaldehyde and ethyl acetate via hydrogenation of acetic acid. The present application claims priority to: Japanese Patent Application No. 2013-165622 filed to Japan on Aug. 8, 2013; Japanese Patent Application No. 2013-169907 filed to Japan on Aug. 19, 2013; Japanese Patent Application No. 2013-175179 and Japanese Patent Application No. 2013-175557 each filed to Japan on Aug. 27, 2013; Japanese Patent Application No. 2013-223356 filed to Japan on Oct. 28, 2013; and Japanese Patent Application No. 2014-081441, Japanese Patent Application No. 2014-081442, Japanese Patent Application No. 2014-081443, Japanese Patent Application No. 2014-081444, and Japanese Patent Application No. 2014-081445 each filed to Japan on Apr. 10, 2014, the entire contents of which are incorporated herein by reference.

### BACKGROUND ART

Acetaldehyde is an intermediate of industrial interest and has been used in a large amount as a starting material typically for ethyl acetate, peracetic acid, pyridine derivative, pentaerythritol, crotonaldehyde, and paraldehyde.

Acetaldehyde has been produced mainly by Wacker oxidation of ethylene. However, improvements in technology have resulted in more inexpensive production of acetic acid from methanol and carbon monoxide. In addition, the ethylene price has increased. These render acetaldehyde production via acetic acid hydrogenation a possible choice. Whether this process is practically performable depends on how high the economic efficiency of the process is increased.

Japanese Unexamined Patent Application Publication (JP-A) No. H11-322658 discloses a method for producing acetaldehyde via acetic acid hydrogenation. With this method, acetic acid is hydrogenated on an iron oxide catalyst containing between 2.5% and 90% by weight palladium in the presence of excess hydrogen. This gives gaseous products including methane, ethane, ethylene, carbon dioxide, acetone, ethanol, ethyl acetate, water, and unreacted acetic acid, in addition to the acetaldehyde main product. The gaseous products are brought into contact with an acetic acid solution in an absorber to separate, via condensation, acetaldehyde, acetone, ethanol, ethyl acetate, water, and acetic acid. The residual hydrogen gas containing non-condensable gases including methane, ethane, ethylene, and carbon dioxide is recycled and reused in the reaction.

A condensate from the absorber is charged into an acetaldehyde recovery column. This gives: offgas that is not condensed with a condenser; the acetaldehyde product from a distillate; and an acetic acid solution from a bottom liquid, where the acetic acid solution contains acetone, ethanol, ethyl acetate, and water. The condensate from the absorber contains dissolved non-condensable gases such as hydrogen, methane, ethane, ethylene, and carbon dioxide. In the acetaldehyde recovery column, the non-condensable gases and acetaldehyde are distributed in the overhead, and the acetaldehyde product as a distillate also contains dissolved non-condensable gases such as hydrogen, methane, ethane, ethylene, and carbon dioxide.

### CITATION LIST

### Patent Literature

PTL 1: JP-A No. H11-322658

### SUMMARY OF INVENTION

### Technical Problem

Disadvantageously, however, the method described in PTL 1, when producing acetaldehyde via acetic acid hydrogenation, requires a complicated process, leads to high cost, and gives the product with a low purity. Accordingly, the present invention has an object to provide a method for producing high-purity acetaldehyde from acetic acid industrially efficiently and inexpensively.

In particular, the recycled gas in the method described in PTL 1 should be partially purged so as to maintain its hydrogen purity at 60% to 95% by mole. When part of the recycled gas is purged so as allow the recycled gas to have a hydrogen purity maintained at 60% to 95% by mole, a large amount of the hydrogen gas that is not used in the reaction is purged and lost, in addition to the non-condensable gases. For example, assume that the selectivity to the non-condensable gases is 5% by mole at an acetaldehyde selectivity of 80% by mole. In this case, maintenance of the hydrogen gas purity at 90% by mole requires purging of the hydrogen gas in an amount corresponding to about 56% by mole of acetaldehyde incident to purging of the non-condensable gases by 5% by mole. This leads to hydrogen cost increase and economic efficiency decrease. Independently, maintenance of the hydrogen gas purity at 60% by mole requires purging of the hydrogen gas in an amount corresponding to only about 9% by mole of acetaldehyde incident to purging of the non-condensable gases by 5% by mole. However, the hydrogen gas partial pressure decreases down to 60% to reduce the reaction rate. The reduction in reaction rate has to be compensated by increasing the reactor volume and/or by increasing the reaction pressure. This also leads to increase in installation cost and decrease in economic efficiency of the process.

Accordingly, the present invention has another object to provide a method for producing acetaldehyde inexpensively via acetic acid hydrogenation without a large purge loss of the hydrogen gas and without significant increase in installation cost.

The acetaldehyde product obtained by the method described in PTL 1 includes non-condensable gases, such as hydrogen, methane, ethane, ethylene, and carbon dioxide, as impurities and is unsatisfactory in quality. A possible solution to this is introduction of an inert gas such as nitrogen to the distillation column to decrease the concentration of the non-condensable gases such as hydrogen, methane, ethane, ethylene, and carbon dioxide. This may reduce the amount of the non-condensable gases to be dissolved in the acetaldehyde product. However, this technique results in a large loss of acetaldehyde with the inert gas, because acetaldehyde has a low boiling point of 21° C.

Accordingly, the present invention has still another object to provide a method for producing acetaldehyde in a high yield via acetic acid hydrogenation, where the product acetaldehyde has a high purity and has extremely low contents of non-condensable gases.

In the method described in PTL 1, the reaction condensate from the absorber includes target acetaldehyde; by-products acetone, ethanol, ethyl acetate, and water; and unreacted acetic acid. The economic efficiency of the process depends on how efficiently, from the reaction condensate, the acetaldehyde product is separated, unreacted acetic acid is recovered, and other valuable substances are separated. PTL 1 describes a process for separating and purifying acetaldehyde, acetic acid, water, ethyl acetate, and acetone from the reaction condensate. This process, however, recovers acetaldehyde and acetic acid, and then separates other components using a stripper and three distillation columns. This requires a complicated process and results in high cost.

Accordingly, the present invention has a further object to provide a method for producing acetaldehyde via acetic acid hydrogenation, where the method separates/purifies acetaldehyde product, unreacted acetic acid, and other valuable substances from a crude reaction liquid simply and highly economically efficiently.

In methods for producing acetaldehyde from acetic acid as in the method described in PTL 1, it is preferred that acetaldehyde is initially separated from the crude reaction liquid via distillation in an acetaldehyde product column, and unreacted acetic acid is subsequently separated via distillation in an acetic acid recovery column. The acetic acid recovery column preferably employs an azeotropic solvent that forms an azeotrope with water. The azeotrope has a lower boiling point as compared with water, and from the azeotrope, water is separable. These allow easy separation of acetic acid and water from each other. In particular, ethyl acetate is preferred as the azeotropic solvent. This is because ethyl acetate exists as a by-product of the acetic acid hydrogenation, and this eliminates a need for an azeotropic solvent recovery step. From the acetic acid recovery column, the overhead product is introduced into a decanter to separate into an upper phase (azeotropic solvent phase) and a lower phase (aqueous phase). The upper-phase distillate is returned into the distillation column, and the lower-phase distillate is fed to a subsequent step. Acetic acid is recovered from the bottom of the acetic acid recovery column. The acetic acid may be recycled to the reaction system. The lower-phase distillate contains by-products acetone, ethanol, and water and, in addition to them, the azeotropic solvent as dissolved. Thus, part of the azeotropic solvent is discharged from the acetic acid recovery column. This requires replenishment of the azeotropic solvent, or recovery of the dissolved azeotropic solvent from the lower-phase distillate and recycling of the recovered azeotropic solvent to the acetic acid recovery column. The replenishment of the azeotropic solvent results in higher cost due to the cost of the azeotropic solvent to be replenished. The recovery of the azeotropic solvent also results in higher cost, because the azeotropic solvent undergoes azeotropic distillation also with ethanol, and separation and recovery of the azeotropic solvent alone from the lower-phase distillate requires a complicated process.

Accordingly, the present invention has still further object to provide a method for producing acetaldehyde via acetic acid hydrogenation, where the method separates, recovers, and recycles the azeotropic solvent inexpensively and simply.

With the method described in PTL 1, the lower-phase distillate is charged into a low-boiling component removal column, and low-boiling components having a lower boiling point as compared with ethyl acetate are recovered from the column top. A bottom liquid from the low-boiling component removal column is charged into an ethanol/ethyl acetate recovery column. At the ethanol/ethyl acetate recovery column, a mixture of ethanol and ethyl acetate is recovered from the column top, and water is discharged from the bottom. Separation of the mixture of ethanol and ethyl acetate, which is obtained from the column top of the ethanol/ethyl acetate recovery column, into ethanol and ethyl acetate requires a complicated process and results in higher cost for yielding ethanol and ethyl acetate obtained as valuable substances. This is because ethanol and ethyl acetate undergoes azeotropy with each other with an azeotropic composition of ethanol to ethyl acetate weight ratio of 31:69.

Accordingly, the present invention has another object to provide a method for utilizing a by-produced mixture of ethanol and ethyl acetate as valuable substances inexpensively and simply, where the mixture is by-produced upon production of acetaldehyde via acetic acid hydrogenation.

In addition, the present invention has still another object to provide a method for producing acetaldehyde and ethyl acetate from acetic acid industrially efficiently.

### Solution to Problem

To achieve the objects, the inventors of the present invention made investigations on selective separation of non-condensable gases from a recycled gas. As a result, the inventors have found that the non-condensable gases are selectively separated from the recycled gas, and the hydrogen gas purge loss is significantly reduced by dissolving the non-condensable gases from the recycled gas into an absorbing liquid, reducing the pressure of the resulting absorbing liquid to strip the non-condensable gases from the absorbing liquid, and recycling the residual liquid after the non-condensable gas stripping to the absorber.

The inventors also made investigations on methods for separating the non-condensable gases from acetaldehyde to achieve the objects. As a result, the inventors have found that high-purity acetaldehyde product devoid of, or approximately devoid of, non-condensable gases is obtained by recovering acetaldehyde in a liquid state from a tray disposed between a starting material feed tray and the column top of an acetaldehyde separating distillation column.

The inventors further made investigations on methods for separating/purifying the acetaldehyde product, unreacted acetic acid, and other valuable substances from a crude reaction liquid to achieve the objects. Consequently, the inventors have found that water, a mixture of ethanol and ethyl acetate, and low-boiling components such as acetone are separated from each other efficiently and inexpensively by using two distillation columns after independent recoveries of acetaldehyde and acetic acid from the crude reaction liquid.

To achieve the objects, the inventors also made investigations on methods for separating/purifying acetaldehyde product, unreacted acetic acid, and other valuable substances from the crude reaction liquid. As a result, the inventors have found a process as follows. In the process, after specific component separation, via distillation using ethyl acetate as an azeotropic solvent, acetic acid is added to part or the whole of a fraction containing ethanol, and the ethanol is esterified in the presence of an acidic catalyst and recycled to an appropriate point in the acetaldehyde production process. The inventors have found that this process allows the azeotropic solvent ethyl acetate to be recycled inexpensively and simply.

The inventors, to achieve the objects, also made investigations on methods for utilizing a mixture of ethanol and ethyl acetate as valuable substances, where the mixture is obtained from the column top (overhead) of the ethanol/ethyl acetate recovery column. Consequently, the inventors have found that a following method eliminates a need for a complicated process for separating ethanol and ethyl acetate from each other. In the method, after specific component separation, via distillation using an azeotropic solvent, acetic acid is added to part or the whole of a mixture of ethanol and ethyl acetate from the column top of the ethanol/ethyl acetate recovery column, and the ethanol is esterified in the presence of an acidic catalyst to yield ethyl acetate.

In addition, to achieve the objects, the inventors made investigations on methods for separating ethanol and an azeotropic solvent from the crude reaction liquid. As a result, the inventors have found a method as follows allows the components such as the azeotropic solvent to be recycled inexpensively and simply. In the method, acetic acid is added to part or the whole of the mixture of ethanol and the azeotropic solvent after the separation of acetaldehyde, unreacted acetic acid, and water therefrom, and the ethanol is esterified into ethyl acetate in the presence of an acidic catalyst. The resulting esterification reaction liquid is subjected to distillation to recover the ethyl acetate from the column top and to recover and recycle the azeotropic solvent from the column bottom.

The present invention has been made based on these findings and further investigations.

Specifically, the present invention relates to followings.

(1) The present invention relates to, according to a first embodiment, a method for producing acetaldehyde via acetic acid hydrogenation. The method includes hydrogenating acetic acid to give a reaction fluid. The reaction fluid is charged into an absorber (absorption column), and, from the reaction fluid, condensed components are absorbed with an absorbing liquid, and non-condensable gases are dissolved into the absorbing liquid. A bottom liquid of the absorber is reduced in pressure (decompressed) to strip the dissolved non-condensable gases from the absorbing liquid, and the residual liquid after the non-condensable gas stripping is recycled to the absorber.

(2) In the acetaldehyde production method according to (1) (the first embodiment), acetaldehyde may be separated from the absorber bottom liquid to leave an aqueous acetic acid solution, and part of the aqueous acetic acid solution may be used as the absorbing liquid in the absorber.

(3) In the acetaldehyde production method according to (1) (the first embodiment), unreacted acetic acid and water may be separated from each other via azeotropic distillation using an azeotropic-solvent-containing liquid, and part of the azeotropic-solvent-containing liquid may be used as the absorbing liquid in the absorber.

(4) In the acetaldehyde production method according to (1) (the first embodiment), a solvent containing 10% by weight or more of an azeotropic solvent may be used as the absorbing liquid in the absorber.

(5) The present invention also relates to, according to a second embodiment, a method for producing acetaldehyde via acetic acid hydrogenation. The method includes hydrogenating acetic acid to give a crude reaction liquid. The crude reaction liquid is subjected to distillation in a distillation column. Upon distillation, acetaldehyde in a liquid phase is recovered from a tray disposed between a crude reaction liquid feed tray and the column top in the distillation column.

(6) The present invention further relates to, according to a third embodiment, a method for producing acetaldehyde via acetic acid hydrogenation. The method includes the steps a), b), c), d-1), and e-1), or includes the steps a), b), c), d-2), and e-2) as follows. In the step a), acetic acid is hydrogenated to give a crude reaction liquid. In the step b), acetaldehyde is separated from the crude reaction liquid via distillation in a first distillation column. In the step c), unreacted acetic acid is separated from the residual liquid after the acetaldehyde separation, via distillation in a second distillation column. In the step d-1), a low-boiling component is separated from the residual liquid after the unreacted acetic acid separation, via distillation in a third distillation column, where the low-boiling component has a lower boiling point as compared with ethyl acetate. In the step e-1), water and a mixture of ethanol and ethyl acetate are separated from the residual liquid after the low-boiling component separation, via distillation in a fourth distillation column. In the step d-2), water is separated from the residual liquid after the unreacted acetic acid separation, via distillation in a third distillation column. In the step e-2), a low-boiling component and a mixture of ethanol and ethyl acetate are separated from the residual liquid after the water separation, via distillation in a fourth distillation column, where the low-boiling component has a lower boiling point as compared with ethyl acetate.

(7) In the acetaldehyde production method according to (6) (the third embodiment), pressures may be controlled upon operation so that the temperature of an overhead vapor of the second distillation column is higher than the bottom temperature of at least one distillation column selected from the group consisting of the first distillation column, the third distillation column, and the fourth distillation column. The overhead vapor of the second distillation column may be used as a heat source for heating at least one distillation column selected from the group consisting of the first distillation column, the third distillation column, and the fourth distillation column.

(8) The present invention also relates to, according to a fourth embodiment, a method for producing acetaldehyde via acetic acid hydrogenation. The method includes the steps a), b), c′), d-1), e-1), f), and g), or includes the steps a), b), c′), d-2), e-2), f), and g), as follows. In the step a), acetic acid is hydrogenated to give a crude reaction liquid. In the step b), acetaldehyde is separated from the crude reaction liquid via distillation in a first distillation column. In the step c′), unreacted acetic acid is separated from the residual liquid after the acetaldehyde separation, via distillation using ethyl acetate as an azeotropic solvent in a second distillation column. In the step d-1), a low-boiling component is separated from the residual liquid after the unreacted acetic acid separation, via distillation in a third distillation column, where the low-boiling component has a lower boiling point as compared with ethyl acetate. In the step e-1), water and a mixture of ethanol and ethyl acetate are separated from the residual liquid after the low-boiling component separation, via distillation in a fourth distillation column. In the step d-2), water is separated from the residual liquid after the unreacted acetic acid separation, via distillation in a third distillation column. In the step e-2), a low-boiling component and a mixture of ethanol and ethyl acetate are separated from the residual liquid after the water separation, via distillation in a fourth distillation column, where the low-boiling component has a lower boiling point as compared with ethyl acetate. In the step f), acetic acid is added to part or the whole of the mixture of ethanol and ethyl acetate and the ethanol is esterified into ethyl acetate in the presence of an acid catalyst. In the step g), ethyl acetate as the azeotropic solvent is recycled.

(9) The present invention also relates to, according to a fifth embodiment, a method for producing acetaldehyde and ethyl acetate via acetic acid hydrogenation. The method includes the steps a), b), c′), d-1), e-1), f), and h), or includes the steps a), b), c′), d-2), e-2), f), and h) as follows. In the step a), acetic acid is hydrogenated to give a crude reaction liquid. In the step b), acetaldehyde is separated from the crude reaction liquid via distillation in a first distillation column. In the step c′), unreacted acetic acid is separated from the residual liquid after the acetaldehyde separation, via distillation using ethyl acetate as an azeotropic solvent in a second distillation column. In the step d-1), a low-boiling component is separated from the residual liquid after the unreacted acetic acid separation, via distillation in a third distillation column, where the low-boiling component has a lower boiling point as compared with ethyl acetate. In the step e-1), water and a mixture of ethanol and ethyl acetate are separated from the residual liquid after the low-boiling component separation, via distillation in a fourth distillation column. In the step d-2), water is separated from the residual liquid after the unreacted acetic acid separation, via distillation in a third distillation column. In the step e-2), a low-boiling component and a mixture of ethanol and ethyl acetate are separated from the residual liquid after the water separation, via distillation in a fourth distillation column, where the low-boiling component has a lower boiling point as compared with ethyl acetate. In the step f), acetic acid is added to part or the whole of the mixture of ethanol and ethyl acetate, and the ethanol is esterified into ethyl acetate in the presence of an acidic catalyst. In the step h), the ethyl acetate is recovered as a product.

(10) The present invention further relates to, according to a sixth embodiment, a method for producing acetaldehyde and ethyl acetate via acetic acid hydrogenation. The method includes the steps a), b), c′), d-1), e-1), f), and i), or includes the steps a), b), c′), d-2), e-2), f), and i) as follows. In the step a), acetic acid is hydrogenated to give a crude reaction liquid. In the step b), acetaldehyde is separated from the crude reaction liquid via distillation in a first distillation column. In the step c′), unreacted acetic acid is separated from the residual liquid after the acetaldehyde separation, via distillation using an azeotropic solvent in a second distillation column. In the step d-1), a low-boiling component is separated from the residual liquid after the unreacted acetic acid separation, via distillation in a third distillation column, where the low-boiling component has a lower boiling point as compared with ethanol. In the step e-1), water and a mixture of ethanol and the azeotropic solvent are separated from the residual liquid after the low-boiling component separation, via distillation in a fourth distillation column. In the step d-2), water is separated from the residual liquid after the unreacted acetic acid separation, via distillation in a third distillation column. In the step e-2), a low-boiling component and a mixture of ethanol and the azeotropic solvent are separated from the residual liquid after the water separation, via distillation in a fourth distillation column, where the low-boiling component has a lower boiling point as compared with ethanol. In the step f), acetic acid is added to part or the whole of the mixture of ethanol and the azeotropic solvent, and the ethanol is esterified into ethyl acetate in the presence of an acidic catalyst to give an esterification reaction liquid. In the step i), the esterification reaction liquid is subjected to distillation in a fifth distillation column to recover the ethyl acetate as an overhead product and to recover the azeotropic solvent as a bottom product, and the recovered azeotropic solvent is recycled.

(11) In the acetaldehyde and ethyl acetate production method according to (10) (the sixth embodiment), the azeotropic solvent may be an ester having a boiling point of 100° C. to 118° C. at normal atmospheric pressure.

(12) The acetaldehyde and ethyl acetate production method according to one of (10) and (11) (the sixth embodiment) may further include controlling pressures upon operation so that the temperature of an overhead vapor of the second distillation column is higher than the bottom temperature of at least one distillation column selected from the group consisting of the first distillation column, the third distillation column, the fourth distillation column, and the fifth distillation column. The overhead vapor of the second distillation column may be used as a heat source for heating at least one distillation column selected from the group consisting of the first distillation column, the third distillation column, the fourth distillation column, and the fifth distillation column.

### Advantageous Effects of Invention

The present invention produces high-purity acetaldehyde from acetic acid inexpensively and industrially efficiently.

With the first embodiment of the present invention, in particular, acetaldehyde is produced from acetic acid inexpensively without a large purge loss of hydrogen gas and without significant increase in installation cost.

With the second embodiment of the present invention, in particular, acetaldehyde is separated in a liquid phase from the crude reaction liquid via distillation in a distillation column at a tray disposed between a crude reaction liquid feed tray and the column top in the distillation column. This configuration gives high-purity acetaldehyde product with small loss of acetaldehyde, where the acetaldehyde product contains none of or, if any, an extremely small amount of non-condensable gases.

With the third embodiment of the present invention for producing acetaldehyde from acetic acid, in particular, acetaldehyde product, unreacted acetic acid, and other valuable substances are simply and highly economically efficiently separated and purified from the crude reaction liquid.

In particular, with the fourth embodiment of the present invention for producing acetaldehyde from acetic acid, by-produced ethanol is converted into ethyl acetate after separation of specific components from the crude reaction liquid. This configuration allows ethyl acetate to be inexpensively and simply recycled to an appropriate point in the acetaldehyde production process.

With the fifth embodiment of the present invention for producing acetaldehyde and ethyl acetate from acetic acid, in particular, the mixture of ethanol and ethyl acetate is converted into ethyl acetate after separation of specific components from the crude reaction liquid. This configuration allows the mixture of ethanol and ethyl acetate to be used as a valuable substance without a complicated process for separating ethanol from ethyl acetate. The configuration also allows acetaldehyde and ethyl acetate to be produced from acetic acid industrially efficiently.

In particular, with the sixth embodiment of the present invention, acetic acid is added to part or the whole of the mixture of ethanol and the azeotropic solvent, the ethanol is esterified into ethyl acetate in the presence of an acidic catalyst, and ethyl acetate and the azeotropic solvent are separated from each other. This configuration allows components such as the azeotropic solvent to be recycled inexpensively and simply.

### BRIEF DESCRIPTION OF DRAWINGS

FIG. 1 is a schematic flow diagram (reaction system 1 (reaction of acetic acid with hydrogen)) illustrating acetaldehyde (and ethyl acetate) production method according to an embodiment of the present invention;

FIG. 2 is a schematic flow diagram (continued from FIG. 1) illustrating an exemplary acetaldehyde production method according to the second embodiment of the present invention;

FIG. 3 is a schematic flow diagram (purification system; continued from FIG. 1) illustrating an exemplary acetaldehyde production method according to the third embodiment of the present invention;

FIG. 4 is a schematic flow diagram (purification system; continued from FIG. 1) illustrating a purification system in another exemplary acetaldehyde production method according to the third embodiment of the present invention;

FIG. 5 is a schematic flow diagram (purification system; continued from FIG. 1) illustrating an exemplary acetaldehyde production method according to the fourth embodiment of the present invention;

FIG. 6 is a schematic flow diagram (purification system; continued from FIG. 1) illustrating another exemplary acetaldehyde production method according to the fourth embodiment of the present invention;

FIG. 7 is a schematic flow diagram (purification system and reaction system 2 (reaction of ethanol with acetic acid); continued from FIG. 1) illustrating an exemplary acetaldehyde and ethyl acetate production method according to the fifth embodiment of the present invention;

FIG. 8 is a schematic flow diagram (purification system and reaction system 2 (reaction of ethanol with acetic acid); continued from FIG. 1) illustrating another exemplary acetaldehyde and ethyl acetate production method according to the fifth embodiment of the present invention;

FIG. 9 is schematic flow diagram illustrating an acetaldehyde production method in examples;

FIG. 10 is a schematic flow diagram illustrating the second embodiment of the present invention in the examples;

FIG. 11 is a schematic flow diagram (purification system; continued from FIG. 1) illustrating an exemplary acetaldehyde and ethyl acetate production method according to the sixth embodiment of the present invention; and

FIG. 12 is a schematic flow diagram (purification system; continued from FIG. 1) illustrating another exemplary acetaldehyde and ethyl acetate production method according to the sixth embodiment of the present invention.

### DESCRIPTION OF EMBODIMENTS

The acetaldehyde production method according to the first embodiment of the present invention is a method for producing acetaldehyde via acetic acid hydrogenation. The method includes, in addition to hydrogenation of acetic acid to give a reaction fluid, an absorbing step and a stripping step. In the absorbing step, the reaction fluid is charged into an absorber. From the reaction fluid, condensed components are absorbed with an absorbing liquid, and non-condensable gases are dissolved into the absorbing liquid. In the stripping step, the bottom liquid of the absorber is reduced in pressure to strip the dissolved non-condensable gases from the absorbing liquid, and the residual liquid after the non-condensable gas stripping is recycled to the absorber.

The acetaldehyde production method according to the second embodiment of the present invention is a method for producing acetaldehyde via acetic acid hydrogenation. The method includes hydrogenating acetic acid to give a crude reaction liquid and subjecting the crude reaction liquid to distillation in a distillation column. Upon the distillation, acetaldehyde in a liquid phase is recovered from a tray disposed between a crude reaction liquid feed tray and the column top in the distillation column.

The acetaldehyde production method according to the third embodiment of the present invention is a method for producing acetaldehyde via acetic acid hydrogenation. The method includes hydrogenating acetic acid to give a crude reaction liquid. From the crude reaction liquid, acetaldehyde is separated via distillation in a first distillation column, and unreacted acetic acid is separated via distillation in a second distillation column. Thereafter, (a) a low-boiling component having a lower boiling point as compared with ethyl acetate, (b) a mixture of ethanol and ethyl acetate, and (c) water are separated from one another using two distillation columns.

The separation of the low-boiling component (a) having a lower boiling point as compared with ethyl acetate, the mixture (b) of ethanol and ethyl acetate, and (c) water using two distillation columns may be performed by one of two processes (1) and (2) as follows. In the process (1) (first process), the low-boiling component (a) is separated from the residual liquid after the unreacted acetic acid separation, via distillation in a third distillation column, where the low-boiling component has a lower boiling point as compared with ethyl acetate. From the residual liquid after the low-boiling component separation, water (c) and the mixture (b) of ethanol and ethyl acetate are separated via distillation in a fourth distillation column. In the process (2) (second process), water (c) is separated from the residual liquid after the unreacted acetic acid separation, via distillation in a third distillation column. From the residual liquid after the water separation, the low-boiling component (a) and the mixture (b) of ethanol and ethyl acetate are separated via distillation in a fourth distillation column, where the low-boiling component has a lower boiling point as compared with ethyl acetate.

The acetaldehyde production method according to the fourth embodiment of the present invention is a method for producing acetaldehyde via acetic acid hydrogenation. The method includes hydrogenating acetic acid to give a crude reaction liquid. From the crude reaction liquid, acetaldehyde, unreacted acetic acid, and water are separated via distillation using ethyl acetate as an azeotropic solvent to leave a fraction containing ethanol. Acetic acid is added to part or the whole of the fraction, the ethanol is esterified into ethyl acetate in the presence of an acidic catalyst, and ethyl acetate as the azeotropic solvent is recycled.

The acetaldehyde and ethyl acetate production method according to the fifth embodiment of the present invention is a method for producing acetaldehyde and ethyl acetate via acetic acid hydrogenation. The method includes hydrogenating acetic acid to give a crude reaction liquid. From the crude reaction liquid, acetaldehyde, unreacted acetic acid, and water are separated via distillation using an azeotropic solvent. The acetaldehyde is recovered as a product. Acetic acid is added to part or the whole of a mixture of ethanol and ethyl acetate after the separation of the acetaldehyde, unreacted acetic acid, and water, the ethanol is esterified into ethyl acetate in the presence of an acidic catalyst, and the ethyl acetate is recovered as a product.

The acetaldehyde and ethyl acetate production method according to the sixth embodiment of the present invention is a method for producing acetaldehyde and ethyl acetate via acetic acid hydrogenation. In the method, acetic acid is hydrogenated to give a crude reaction liquid. From the crude reaction liquid, acetaldehyde, unreacted acetic acid, and water are separated via distillation using an azeotropic solvent. The acetaldehyde is recovered as a product. Acetic acid is added to part or the whole of a mixture of ethanol and the azeotropic solvent after the separation of the acetaldehyde, unreacted acetic acid, and water, and the ethanol is esterified into ethyl acetate in the presence of an acidic catalyst to give an esterification reaction liquid. The esterification reaction liquid is subjected to distillation. The ethyl acetate is recovered from the column top, and the azeotropic solvent is recovered from the column bottom and recycled.

The present invention will be illustrated in detail below, with reference to the attached drawings as needed.

Reaction System 1 (Reaction of Acetic Acid with Hydrogen)

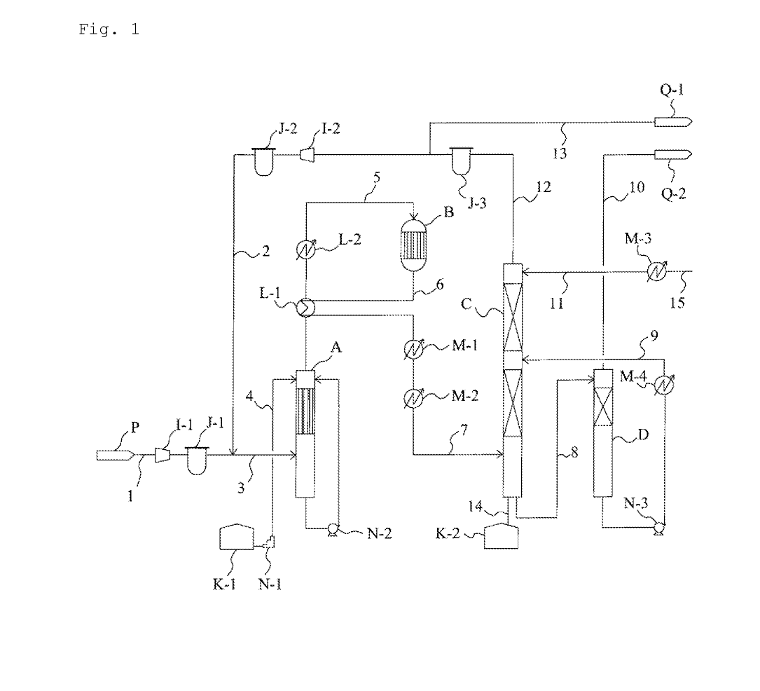

FIG. 1 illustrates an embodiment in which hydrogen gas is fed from a hydrogen installation P via a line 1, compressed with a compressor I-1, fed through a buffer tank J-1, merged with a recycled gas from a line 2, and is charged via a line 3 into an evaporator A (acetic acid evaporator). Acetic acid is fed from an acetic acid tank K-1 via a line 4 to the evaporator An using a pump N-1 to vaporize acetic acid. The vaporized acetic acid is, together with the hydrogen gas, heated with heat exchangers (heaters) L-1 and L-2 and charged via a line 5 into a catalyst-packed reactor B. The evaporator A is equipped with a circulating pump N-2. The acetic acid is hydrogenated in the reactor B to give main product acetaldehyde, as well as non-condensable products methane, ethane, ethylene, and carbon dioxide; and condensable products acetone, ethanol, ethyl acetate, and water.

The acetic acid hydrogenation may be performed by a known technique. For example, acetic acid is allowed to react with hydrogen in the presence of a catalyst. The catalyst is not limited, as long as one capable of forming acetaldehyde via acetic acid hydrogenation. Examples of the catalyst include, but are not limited to, metal oxides such as iron oxides, germanium oxides, tin oxides, vanadium oxides, and zinc oxide. The catalyst may also be one including any of such metal oxides combined with any of noble metals such as palladium and platinum. In this case, the amount of the noble metal is typically about 0.5% to about 90% by weight based on the total amount of the catalyst. Among them, preferred are catalysts that include iron oxide combined with a noble metal such as palladium and/or platinum. The catalyst may have undergone a reduction treatment typically by bringing into contact with hydrogen, before use for the acetic acid hydrogenation. The reduction treatment may be performed typically at a temperature of 50° C. to 500° C. and a pressure of 0.1 to 5 MPa.

The reaction (hydrogenation) may be performed at a temperature of typically 250° C. to 400° C., and preferably 270° C. to 350° C. The reaction, if performed at an excessively low temperature, may lead to increased by-production typically of ethanol; and, if performed at an excessively high temperature, may lead to increased by-production typically of acetone. In any of these cases, the acetaldehyde may tend to be produced with a lower selectivity. The reaction may be performed at normal atmospheric pressure, under reduced pressure, or under pressure (under a load) without limitation in terms of pressure, but is performed at a pressure of generally from 0.1 to 10 MPa, and preferably from 0.1 to 3 MPa.

Hydrogen and acetic acid may be fed to the reactor at a hydrogen to acetic acid ratio (mole ratio) of generally 0.5 to 50, and preferably 2 to 25.

In the reactor, acetic acid is desirably converted with a conversion of 50% or less (e.g., 5% to 50%). Acetic acid, if converted with a conversion of greater than 50%, may cause by-products (e.g., ethanol and ethyl acetate) to more readily form and cause the acetaldehyde product to form with a lower selectively. To eliminate or minimize this problem, the residence time and the hydrogen space velocity in the reactor are preferably adjusted so that the acetic acid conversion is 50% or less.

The reaction between acetic acid and hydrogen yields a gaseous reaction products mainly including unconverted acetic acid, unconverted hydrogen, acetaldehyde, water, and other products (such as ethanol, ethyl acetate, and acetone) formed via the reaction, as described above.

The gaseous reaction products may be separated into non-condensable gases and condensable components. The condensable components may be handled as a crude reaction liquid (crude reaction mixture). The gaseous reaction products may be separated into non-condensable gases and condensable components typically, but not limitatively, by charging the reaction fluid from the acetic acid hydrogenation into an absorber, and absorbing the condensed components from the reaction fluid with an absorbing liquid (absorbing step). The condensable components absorbed by the absorbing liquid (a mixture of the condensable components and the absorbing liquid) is also included in the “crude reaction liquid” in the present invention. In the absorbing step, part of the non-condensable gases is dissolved in the absorbing liquid. Even in this case, hydrogen is efficiently separated from other non-condensable gas components by providing a stripping step. In the stripping step, the bottom liquid of the absorber is reduced in pressure to strip (desorb) dissolved non-condensable gases from the absorbing liquid, and the residual liquid after the non-condensable gas stripping is recycled to the absorber.

In the absorbing step in the present invention, the reaction fluid from the acetic acid hydrogenation is charged into the absorber and, from the reaction fluid, condensed components are absorbed with an absorbing liquid, and non-condensable gases are dissolved into the absorbing liquid. The absorbing step is generally performed by supplying the absorbing liquid and the reaction fluid from the reaction step into the absorber, and bringing them into contact with each other in the absorber. Examples of the absorber include, but are not limited to, publicly or commonly known gas absorbers with configurations typically of packed columns, plate columns, spray columns, and wetted wall columns.

In the stripping step in the present invention, the bottom liquid from the absorber is reduced in pressure to strip (desorb) dissolved non-condensable gases from the absorbing liquid, and the residual liquid after the non-condensable gas stripping is recycled to the absorber. The stripping step is generally performed by supplying the absorber bottom liquid from the absorbing step to a stripper under reduced pressure to strip non-condensable gases, where the “bottom liquid” refers to the absorbing liquid after absorption and dissolution of condensed components and non-condensable gases. Examples of the stripper include, but are not limited to, publicly or commonly known gas strippers with configurations typically of packed columns, plate columns, spray columns, wetted wall columns, and gas-liquid separators.

In the embodiment illustrated in FIG. 1, the reaction fluid discharged from the reactor B is fed via a line 6 through the heat exchanger L-1, and cooled with heat exchangers (condensers) M-1 and M-2, and charged via a line 7 into a lower portion of an absorber C. A recycled liquid as an absorbing liquid is charged via a line 9 into the absorber C. The “recycled liquid” herein refers to a bottom liquid from an after-mentioned stripper D. The recycled liquid mainly absorbs and dissolves therein hydrogen, methane, ethane, ethylene, and carbon dioxide, which are non-condensable gases. An upper-phase distillate from an after-mentioned acetic acid recovery column F is charged as an absorber supply liquid via a line 11 into the absorber C. The “absorber supply liquid” refers to another absorbing liquid than the recycled liquid. The upper-phase distillate is rich in an azeotropic solvent that undergoes azeotropy with water. The absorber supply liquid absorbs acetaldehyde together with the non-condensable gases, where the acetaldehyde is a low-boiling condensable component. The upper-phase distillate from the acetic acid recovery column F is fed via a line 15 through a cooler M-3 to the line 11. A bottom liquid from the stripper D (recycled liquid) (line 9) is preferably charged into a middle portion of the absorber C; and the upper-phase distillate from the acetic acid recovery column F (absorber supply liquid; line 11) is preferably charged into an upper portion of the absorber C, although these liquids may be charged into the absorber C at appropriately selected positions in consideration of the absorption efficiency of the acetaldehyde and non-condensable gases.

A bottom liquid from the absorber C is divided into a line 14 and a line 8. The line 14 leads to a purification process. The line 8 leads to the stripper D. The bottom liquid in the line 14 is stored as a crude reaction liquid in a crude reaction liquid tank K-2 and is subjected to the purification process. The liquid in the line 8 is decompressed in the stripper D, and, from the liquid, hydrogen, methane, ethane, ethylene, and carbon dioxide, which are non-condensable gases dissolved in the absorbing liquid, are stripped via a line 10. The residual liquid after the non-condensable gas stripping is recycled via the line 9 to the absorber C. The line 10 is linked to a vent Q-2. It is accepted that, for example, the whole quantity of the bottom liquid from the absorber C is charged into the stripper D, part of the residual liquid after the non-condensable gas stripping is recycled to the absorber, and the remainder of the liquid is subjected as a crude reaction liquid to the purification process (see the working examples).

According to the present invention, the non-condensable gases are dissolved in the absorbing liquid, and the absorber bottom liquid is then decompressed to strip the dissolved non-condensable gases from the absorbing liquid. This configuration allows hydrogen to be efficiently separated from other non-condensable gases, because of difference in solubility between hydrogen and the other non-condensable gases. For example, hydrogen and methane have solubilities in ethyl acetate of respectively 0.01 NL/L and 0.48 NL/L at 30° C. and at a partial pressure of 1 atm (one atmospheric pressure). This indicates that methane is dissolved in ethyl acetate in an amount 48 times greater than hydrogen. In addition, the present invention recycles the residual liquid after the non-condensable gas stripping to the absorber. This configuration allows other non-condensable gases than hydrogen gas to be efficiently absorbed/dissolved and results in significant reduction of hydrogen gas purge loss.

Non-condensable gases that have not been absorbed by or dissolved in the absorbing liquid in the absorber C are discharged from the column top of the absorber C, fed via a line 12 through a buffer tank J-3, pressurized with a compressor I-2, fed through a buffer tank J-2 via the line 2, merged with the hydrogen gas in the line 1, and fed via the line 3 to the evaporator A. The non-condensable gases are purged via a line 13 through a vent Q-1 as needed.

In the embodiment, acetaldehyde is separated from the bottom liquid of the absorber C to leave a liquid mixture containing acetic acid and water (aqueous acetic acid solution). The liquid mixture is subjected to an acetic acid recovery step in the acetic acid recovery column F to recover acetic acid from the liquid mixture. The acetic acid recovery step is the step of separating unreacted acetic acid and by-produced water from each other via azeotropic distillation. Of distillates from the acetic acid recovery column F, an upper-phase distillate is used as the absorbing liquid for use in the absorber C. The upper-phase distillate is a liquid rich in the azeotropic solvent, where the azeotropic solvent is a solvent that undergoes azeotropy with water. The lower-phase distillate of the acetic acid recovery column F is rich in water and forms an aqueous phase.

The absorbing liquid to be charged into the absorber C may include the absorber C bottom liquid (recycled liquid) alone, but preferably includes an absorbing liquid devoid of, or approximately devoid of, acetaldehyde. This is preferred for higher acetaldehyde recovery, because the absorber C bottom liquid is rich in acetaldehyde which has a low boiling point of 21° C. Preferred examples of the absorbing liquid include, but are not limited to, an azeotropic-solvent-containing liquid for use in separation of unreacted acetic acid and by-produced water from each other via azeotropic distillation, as in the embodiment, where the azeotropic-solvent-containing liquid is an upper-phase liquid rich in the azeotropic solvent, and where the upper-phase liquid is obtained via separation of distillates from the acetic acid recovery column F using a decanter; and aqueous acetic acid solutions (liquid mixtures containing acetic acid and water) such as a liquid derived from the absorber C bottom liquid after separation of acetaldehyde from the bottom liquid. The mixture of acetic acid and water is also exemplified by the bottom liquid from an after-mentioned acetaldehyde product column E. The absorbing liquid is preferably a liquid containing ethyl acetate in a content of 10% by weight or more (more preferably 30% by weight or more, furthermore preferably 50% by weight or more, and particularly preferably 75% by weight or more).

The azeotropic-solvent-containing liquid, when used as the absorbing liquid, may contain the azeotropic solvent in a content of typically 10% by weight or more, preferably 30% by weight or more, more preferably 50% by weight or more, and particularly preferably 75% by weight or more. The aqueous acetic acid solution, when used as the absorbing liquid, may contain acetic acid in a content of typically 10% to 95% by weight, preferably 50% to 90% by weight, and more preferably 60% to 80% by weight.

The azeotropic solvent forms an azeotrope (azeotropic mixture) with water to have a lower boiling point; and the resulting azeotrope is separated into the azeotropic solvent and water. This allows easy separation of acetic acid and water from each other. Examples of the azeotropic solvent include, but are not limited to, esters such as isopropyl formate, propyl formate, butyl formate, isoamyl formate, ethyl acetate, isopropyl acetate, propyl acetate, butyl acetate, methyl propionate, ethyl propionate, methyl butyrate, ethyl butyrate, and isopropyl butyrate; ketones such as methyl ethyl ketone, methyl propyl ketone, methyl isobutyl ketone, diethyl ketone, and ethyl propyl ketone; aliphatic hydrocarbons such as pentane, hexane, and heptane; alicyclic hydrocarbons such as cyclohexane, methylcyclohexane, and dimethylcyclohexane; and aromatic hydrocarbons such as benzene and toluene.

Among them, ethyl acetate is preferred as the azeotropic solvent, because ethyl acetate is present as a by-product of the acetic acid hydrogenation, and this eliminates or minimizes a need for the azeotropic solvent recovery step.

Esters having boiling points of 100° C. to 118° C. at normal atmospheric pressure are also preferred as the azeotropic solvent. Examples of the esters include, but are not limited to, propyl acetate (boiling point: 102° C.), isobutyl acetate (boiling point: 117° C.), sec-butyl acetate (boiling point: 112° C.), isopropyl propionate (boiling point: 110° C.), methyl butyrate (boiling point: 102° C.), and ethyl isobutyrate (boiling point: 110° C.). The esters having boiling points of 100° C. to 118° C. are preferred for the following reasons. The esters form, with water, azeotropes having high water proportions and having lower boiling points as compared with acetic acid. This allows easier separation of acetic acid and water from each other in the acetic acid recovery column F. In addition, the esters do not undergo azeotropy with ethanol or form, if any, azeotropes with ethanol having low ethanol proportions. The esters, when used as the azeotropic solvent, are therefore relatively easily separated and recovered as the azeotropic solvent.

Methane as a principal component of the non-condensable gases is dissolved more readily in an azeotropic solvent having low polarity than in an aqueous acetic acid solution having high polarity. The azeotropic solvent is therefore suitable as the absorbing liquid for the non-condensable gases. Ethyl acetate is therefore also suitable as the absorbing liquid.

The ratio (weight ratio) in amount of the absorber supply liquid (line 11) to the reaction fluid (line 7) each fed to the absorber C is typically 0.1 to 10, and preferably 0.3 to 2. The ratio (weight ratio) in amount of the recycled liquid (line 9) to the reaction fluid (line 7) each fed to the absorber C is typically 0.05 to 20, and preferably 0.1 to 10.

The absorber C may include 1 to 20 theoretical trays, and preferably 3 to 10 theoretical trays. In the absorber C, the temperature is typically 0° C. to 70° C., and the pressure is typically 0.1 to 5 MPa (absolute pressure).

The temperature of the stripper D is typically 0° C. to 70° C. The pressure in the stripper D is not limited, as long as being lower than the pressure of the absorber C, but is typically 0.05 to 4.9 MPa (absolute pressure). The pressure of the stripper D may be lower than the pressure of the absorber C by typically 0.05 to 4.9 MPa, and preferably 0.5 to 2 MPa, while the difference in pressure may be selected as appropriate from the viewpoints of non-condensable gas stripping efficiency and acetaldehyde loss reduction.

Purification Process (Purification System)

The crude reaction liquid from the reaction system is subjected to a purification process (purification system) to yield acetaldehyde as a product. The purification process also allows recovery of unreacted acetic acid and by-produced components and recycling of one or more of them to the reactor as needed. The purification process may include one or more steps selected typically from an acetaldehyde purification step, an acetic acid recovery step, a low-boiling component removal step, and an ethanol/ethyl acetate recovery step. In the acetaldehyde purification step, acetaldehyde is separated and recovered from the crude reaction liquid. In the acetic acid recovery step, unreacted acetic acid and water are separated, via azeotropic distillation, from the residual liquid after the acetaldehyde separation, and acetic acid is recovered. In the low-boiling component removal step, a low-boiling component or components are separated and removed from the residual liquid after the acetic acid separation. In the ethanol/ethyl acetate recovery step, ethanol and/or ethyl acetate is separated and recovered from the residual liquid after the low-boiling component separation and removal.

The acetaldehyde purification step may be performed typically in the following manner. The crude reaction liquid is charged into a distillation column (acetaldehyde product column), and acetaldehyde is separated and recovered from the column top. From the column bottom, an aqueous acetic acid solution is discharged. The aqueous acetic acid solution contains unreacted acetic acid and by-produced water (and generally further contains other products such as ethanol and ethyl acetate).

The purification system according to an embodiment of the present invention includes an acetaldehyde purification step and an acetic acid recovery step. In the acetaldehyde purification step, acetaldehyde is separated from the crude reaction liquid via distillation in a first distillation column, where the crude reaction liquid is derived from the acetic acid hydrogenation. In the acetic acid recovery step, unreacted acetic acid is separated, via distillation in a second distillation column, from the residual liquid after the acetaldehyde separation.

The acetaldehyde purification step may be performed typically in the following manner. The crude reaction liquid is charged into the first distillation column (acetaldehyde product column), and acetaldehyde is separated and recovered from the column top. From the column bottom, an aqueous acetic acid solution is discharged. The aqueous acetic acid solution contains unreacted acetic acid and by-produced water (and generally further contains other products such as ethanol and ethyl acetate).

The column top pressure of the acetaldehyde product column is generally 0.1 MPa or more, and preferably 0.5 to 2 MPa and is, in terms of gauge pressure, generally 0.0 MPaG or more, and preferably 0.4 to 1.9 MPaG. The acetaldehyde product column may include typically 10 to 50 theoretical trays, and preferably 20 to 40 theoretical trays. The distillation may be performed at normal atmospheric pressure, under reduced pressure, or under pressure (under a load) without limitation.

In the acetic acid recovery step, the bottoms (bottom liquid) from the acetaldehyde product column are charged into the second distillation column (acetic acid recovery column), and a liquid containing an azeotropic solvent (solvent that undergoes azeotropy with water) is fed through the column top into the acetic acid recovery column. The overhead product is introduced into a decanter to separate into an upper phase (organic phase) and a lower phase (aqueous phase). Upon the separation in the decanter, ethyl acetate or the azeotropic solvent may be replenished. Part of the upper-phase distillate is returned to the distillation column, part of which may be used as the absorbing liquid in the absorber, as described above. The lower-phase distillate and the remainder of the upper-phase distillate are fed typically to an after-mentioned low-boiling component removal column.

Acetic acid is recovered from the column bottom of the acetic acid recovery column. The acetic acid may be recycled to the reaction system.

The acetic acid recovery column may include typically 10 to 50 theoretical trays, and preferably 10 to 30 theoretical trays. The distillation may be performed at normal atmospheric pressure, under reduced pressure, or under pressure (under a load) without limitation.

In the low-boiling component removal step, the lower-phase distillate and part of the upper-phase distillate from the acetic acid recovery column are charged into a distillation column (low-boiling component removal column). A low-boiling component or components are recovered from the column top, and a liquid containing ethanol, ethyl acetate, and water is discharged from the column bottom. The bottom liquid is fed typically to an after-mentioned ethanol/ethyl acetate recovery column.

The low-boiling component removal column may include typically 10 to 50 theoretical trays, and preferably 20 to 40 theoretical trays. The distillation may be performed at normal atmospheric pressure, under reduced pressure, or under pressure (under a load) without limitation.

In the ethanol/ethyl acetate recovery step, the bottom liquid from the low-boiling component removal column is charged into the ethanol/ethyl acetate recovery column. Ethanol and ethyl acetate are recovered from the column top, and water is discharged from the column bottom.

The ethanol/ethyl acetate recovery column may include typically 5 to 50 theoretical trays, and preferably 10 to 20 theoretical trays. The distillation may be performed at normal atmospheric pressure, under reduced pressure, or under pressure (under a load) without limitation.

According to the second embodiment of the present invention, the crude reaction liquid is charged into the distillation column (acetaldehyde product column), and liquid-phase (liquid-form) acetaldehyde is recovered from a tray (also including a first tray from the column top (uppermost tray)) disposed between the distillation column crude reaction liquid feed tray and the column top in the distillation column, in the acetaldehyde purification step. This configuration yields high-purity acetaldehyde product that contains none of or, if any, an extremely small amount of non-condensable gases.

The acetaldehyde product column may be any of a plate column and a packed column. In the case of the plate column, the trays are not limited in their structure and may be selected typically from bubble-cap trays, perforated trays, and valve trays. In the case of the packed column, the packing material may be any of stacked packings and dumped packings. The column may include trays in a number not limited, as long as the acetaldehyde product is obtained in a needed yield with needed quality, but may include typically about 10 to about 50 theoretical trays. The column, if including trays in an excessively small number, may cause acetaldehyde to be produced in a lower yield and/or with lower quality. Alternatively, to make up for the lowered yield and/or lowered quality so as to obtain the product in a predetermined yield with predetermined quality, larger reflux should be performed, and this leads to a larger amount of heat necessary for the separation.

The position of the tray at which the acetaldehyde product is side-cut is higher than a tray at which the crude reaction liquid is fed, and is lower than the column top. With approaching the feed tray, the product may tend to be mixed with larger amounts of high-boiling substances such as acetone, ethyl acetate, and water. To eliminate or minimize this, the acetaldehyde product is desirably side-cut at a position corresponding approximately to the fifth tray from the uppermost tray (first tray).

An aqueous acetic acid solution is discharged from the distillation column bottom. The aqueous acetic acid solution contains unreacted acetic acid and by-produced water (and generally further contains other products such as ethanol and ethyl acetate).

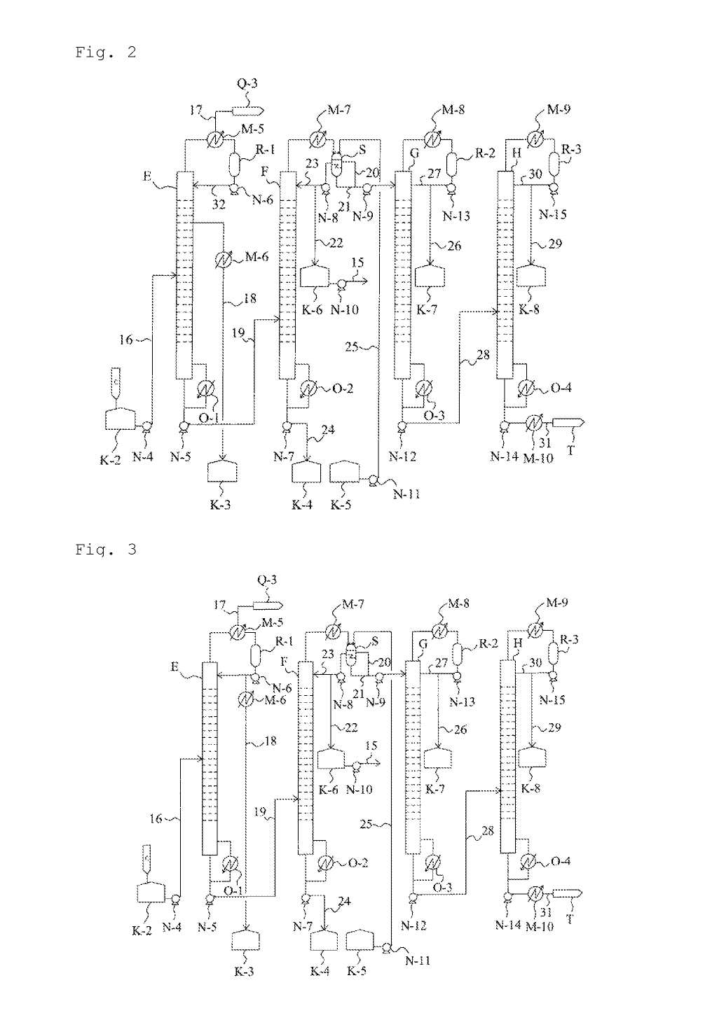

In the embodiment illustrated in FIG. 2, the crude reaction liquid is charged from the crude reaction liquid tank K-2 using a pump N-4 via a line 16 into the first distillation column (acetaldehyde product column) E. From the first distillation column (acetaldehyde product column) E, non-condensable gases are purged via a line 17 and are condensed with a condenser M-5. The resulting liquid is refluxed via a line 32 to the distillation column. Acetaldehyde in a liquid phase is drawn out, via a line 18, from a tray disposed between a feed tray and the column top in the acetaldehyde product column E, where the crude reaction liquid is fed to the feed tray via the line 16. The acetaldehyde is cooled with a cooler M-6 and is stored in an acetaldehyde product tank K-3. A bottom liquid from the first distillation column (acetaldehyde product column) E is fed via a line 19 to the acetic acid recovery column F. The system in the embodiment also includes a receiver R-1, pumps N-5 and N-6, a vent Q-3, and a reboiler O-1.

At the acetic acid recovery column F, an azeotropic-solvent-containing liquid is charged via a line 23 into the column top. Unreacted acetic acid is recovered as a bottom liquid via a line 24, stored in a recovered acetic acid tank K-4, and recycled to the reaction system. Acetone, ethanol, ethyl acetate, water, and the azeotropic solvent are distilled from the column top of the acetic acid recovery column F and separated with a decanter S into a lower-phase water and an upper-phase liquid. The lower-phase water and part of the upper-phase liquid (as needed) are charged respectively via a line 21 and a line 20 into a low-boiling component removal column G. The azeotropic solvent (such as ethyl acetate) is fed from an azeotropic solvent tank K-5 via a line 25 to the decanter S. Part of the upper-phase liquid in the decanter S is fed via the line 22 and stored in an absorbing liquid tank K-6 and is charged, via the line 15 and the line 11, also into the absorber C to absorb acetaldehyde, as described above. Part of the upper-phase liquid in the decanter S is returned via the line 23 into the distillation column. The system also includes a condenser M-7, pumps N-7, N-8, N-9, N-10, and N-11, and a reboiler O-2.

Low-boiling components such as acetone are distilled from the column top of the low-boiling component removal column G via a line 26, and a bottom liquid is charged via a line 28 into an ethanol/ethyl acetate recovery column H. Part of the overhead products is refluxed via a line 27 into the distillation column. The system also includes a condenser M-8, a receiver R-2, pumps N-12 and N-13, a reboiler O-3, and a low-boiling component tank K-7.

At the ethanol/ethyl acetate recovery column H, ethanol, ethyl acetate (by-product), and the azeotropic solvent (such as ethyl acetate) are recovered from the column top via a line 29, and a bottom liquid (water) is discharged via a line 31. The system also includes a condenser M-9 and a cooler M-10, a receiver R-3, pumps N-14 and N-15, a reboiler O-4, and a recovered ethanol/ethyl acetate tank K-8.

The mixture of ethanol, ethyl acetate, and the azeotropic solvent from the line 29 may be further subjected to distillation and/or extraction according to necessity for separation.

A liquid derived from the crude reaction liquid after the separation of acetaldehyde and unreacted acetic acid contains (a) acetone and other low-boiling components having a lower boiling point as compared with ethyl acetate; (b) ethanol and ethyl acetate; and (c) water. Examples of a process for separating these components include, but are not limited to, following first and second processes.

First Process

In the first process (1) includes the steps of separating the low-boiling component(s) (a) having a lower boiling point as compared with ethyl acetate from the residual liquid after the unreacted acetic acid separation, via distillation in a third distillation column; and separating the mixture (b) of ethanol and ethyl acetate and the water (c) from the residual liquid after the low-boiling component separation, via distillation in a fourth distillation column. More specifically the low-boiling component(s) (a) having a lower boiling point as compared with ethyl acetate is initially separated from the residual liquid after the unreacted acetic acid separation, via distillation in the third distillation column (low-boiling component removal step), and subsequently, the water (c) and the mixture (b) of ethanol and ethyl acetate are separated from the residual liquid after the low-boiling component separation, via distillation in the fourth distillation column (ethanol/ethyl acetate recovery step).

In the low-boiling component removal step, the lower-phase distillate from the acetic acid recovery column, and part of the upper-phase distillate (as needed) are charged into the third distillation column (low-boiling component removal column). The low-boiling component(s) is recovered from the column top, and a liquid containing ethanol and ethyl acetate is discharged from the column bottom. The bottom liquid is fed to the after-mentioned fourth distillation column (ethanol/ethyl acetate recovery column).

The third distillation column (low-boiling component removal column) may include typically 10 to 50 theoretical trays, and preferably 20 to 40 theoretical trays. The distillation may be performed at normal atmospheric pressure, under reduced pressure, or under pressure (under a load) without limitation.

In the ethanol/ethyl acetate recovery step, the bottom liquid from the third distillation column (low-boiling component removal column) is charged into the fourth distillation column (ethanol/ethyl acetate recovery column). Ethanol and ethyl acetate are recovered from the column top, and water is discharged from the column bottom.

The fourth distillation column (ethanol/ethyl acetate recovery column) may include typically 5 to 50 theoretical trays, and preferably 10 to 20 theoretical trays. The distillation may be performed at normal atmospheric pressure, under reduced pressure, or under pressure (under a load) without limitation.

Second Process

The second process (2) includes the steps of separating the water (c) from the residual liquid after the unreacted acetic acid separation, via distillation in a third distillation column; and separating the low-boiling component(s) (a) having a lower boiling point as compared with ethyl acetate and the mixture (b) of ethanol and ethyl acetate from the residual liquid after the water separation, via distillation in a fourth distillation column. More specifically, the water (c) is initially separated from the liquid after the unreacted acetic acid separation, via distillation in the third distillation column (water separation step). Subsequently, the low-boiling component(s) (a) having a lower boiling point as compared with ethyl acetate and the mixture (b) of ethanol and ethyl acetate are separated from the residual liquid after the water separation, via distillation in the fourth distillation column (low-boiling component recovery step).

In the water separation step, the lower-phase distillate from the second distillation column (acetic acid recovery column) and part of the upper-phase distillate (as needed) are charged into the third distillation column (water separation column). Ethanol, ethyl acetate, and low-boiling component(s) having a lower boiling point as compared with ethyl acetate are distilled from the column top, and water is discharged from the column bottom. The overhead products are fed to the after-mentioned fourth distillation column (low-boiling component recovery column).

The third distillation column (water separation column) may include typically 5 to 50 theoretical trays, and preferably 10 to 20 theoretical trays. The distillation may be performed at normal atmospheric pressure, under reduced pressure, or under pressure (under a load) without limitation.

In the low-boiling component recovery step, the overhead products from the third distillation column (water separation column) are charged into the fourth distillation column (low-boiling component recovery column). Acetone and any other low-boiling components having a lower boiling point as compared with ethyl acetate are recovered from the column top, and a liquid mixture of ethanol and ethyl acetate is recovered from the column bottom.

The fourth distillation column (low-boiling component recovery column) may include typically 10 to 50 theoretical trays, and preferably 20 to 40 theoretical trays. The distillation may be performed at normal atmospheric pressure, under reduced pressure, or under pressure (under a load) without limitation.

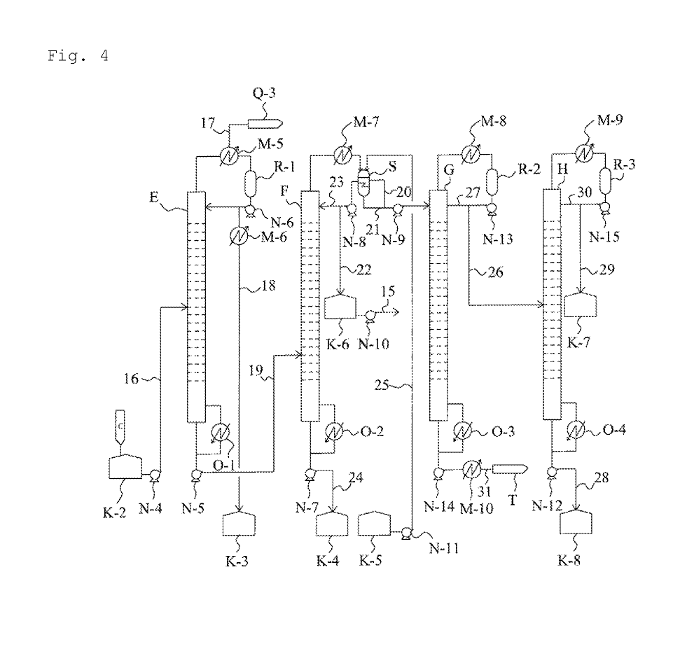

FIG. 3 is a schematic flow diagram illustrating a purification system according to the third embodiment of the present invention, where the purification system includes the first process; and FIG. 4 is a schematic flow diagram illustrating another purification system according to the third embodiment of the present invention, where the purification system includes the second process.

In the embodiment illustrated in FIG. 3, the crude reaction liquid is charged from the crude reaction liquid tank K-2 using the pump N-4 via the line 16 to the first distillation column (acetaldehyde product column) E. From the column top of the first distillation column (acetaldehyde product column) E, non-condensable gases are purged via the line 17, and acetaldehyde product is distilled via the line 18. A bottom liquid from the first distillation column (acetaldehyde product column) E is fed via the line 19 to the second distillation column (acetic acid recovery column) F. The system also includes the condensers M-5 and M-6, the receiver R-1, the pumps N-5 and N-6, the vent Q-3, the reboiler O-1, and the acetaldehyde product tank K-3.

In the second distillation column (acetic acid recovery column) F, an azeotropic-solvent-containing liquid is charged via the line 23 into the column top; and unreacted acetic acid is recovered as a bottom liquid via the line 24, stored in the recovered acetic acid tank K-4, and recycled to the reaction system. Acetone, ethanol, ethyl acetate, water, and the azeotropic solvent are distilled from the column top of the second distillation column (acetic acid recovery column) F, are separated with the decanter S into an upper-phase liquid and lower-phase water. Part of the upper-phase liquid (as needed) and the lower-phase water are charged respectively via the line 20 and the line 21 into the third distillation column (low-boiling component removal column) G. To the decanter S, the azeotropic solvent (such as ethyl acetate) is fed from the azeotropic solvent tank K-5 via the line 25. Part of the upper-phase liquid from the decanter S is stored via the line 22 into the absorbing liquid tank K-6, and is charged, via the line 15 and the line 11, also into the absorber C to absorb acetaldehyde, as described above. Part of the upper-phase liquid is refluxed from the decanter S via the line 23 into the distillation column. The system also includes the condenser M-7, the pumps N-7, N-8, N-9, N-10, and N-11, and the reboiler O-2.

Low-boiling components such as acetone are distilled from the column top of the third distillation column (low-boiling component removal column) G via the line 26, and a bottom liquid is discharged from the column bottom and charged via the line 28 into a fourth distillation column (ethanol/ethyl acetate recovery column) H. Part of the overhead products is refluxed via the line 27 into the distillation column. The system also includes the condenser M-8, the receiver R-2, the pumps N-12 and N-13, the reboiler O-3, and the low-boiling component tank K-7.

At the fourth distillation column (ethanol/ethyl acetate recovery column) H, ethanol, ethyl acetate (by-product), and the azeotropic solvent (such as ethyl acetate) are recovered from the column top via the line 29, and a bottom liquid (water) is discharged via the line 31. Part of the overhead products is refluxed via the line 30 into the distillation column. The system also includes the condenser M-9 and the cooler M-10, the receiver R-3, the pumps N-14 and N-15, the reboiler O-4, and the recovered ethanol/ethyl acetate tank K-8.

The mixture of ethanol, ethyl acetate, and the azeotropic solvent from the line 29 may be further subjected to distillation and/or extraction to separate from each other according to necessity.

In the embodiment illustrated in FIG. 4, the crude reaction liquid is charged from the crude reaction liquid tank K-2 using the pump N-4 via the line 16 into the first distillation column (acetaldehyde product column) E. From the column top of the first distillation column (acetaldehyde product column) E, non-condensable gases are purged via the line 17, and acetaldehyde product is distilled via the line 18. A bottom liquid from the first distillation column (acetaldehyde product column) E is fed via the line 19 to the second distillation column (acetic acid recovery column) F. The system also includes the condensers M-5 and M-6, the receiver R-1, the pumps N-5 and N-6, the vent Q-3, the reboiler O-1, and the acetaldehyde product tank K-3.

In the second distillation column (acetic acid recovery column) F, an azeotropic-solvent-containing liquid is charged via the line 23 into the column top. Unreacted acetic acid is recovered as a bottom liquid via the line 24, stored in the recovered acetic acid tank K-4, and recycled to the reaction system. Acetone, ethanol, ethyl acetate, water, and the azeotropic solvent are distilled from the column top of the second distillation column (acetic acid recovery column) F and separated with the decanter S into an upper-phase liquid and lower-phase water. Part of the upper-phase liquid (as needed) and the lower-phase water are charged respectively via the line 20 and the line 21 into a third distillation column G. The third distillation column G in this case functions as a water separation column. The azeotropic solvent (such as ethyl acetate) is supplied from the azeotropic solvent tank K-5 via the line 25 to the decanter S. Part of the upper-phase liquid from the decanter S is stored via the line 22 in the absorbing liquid tank K-6 and also charged via the line 15 and the line 11 into the absorber C to absorb acetaldehyde, as described above. Another part of the upper-phase liquid is refluxed from the decanter S via the line 23 into the distillation column. The system also includes the condenser M-7, the pumps N-7, N-8, N-9, N-10, and N-11, and the reboiler O-2.

From the column top of the third distillation column (water separation column) G, low-boiling components (such as acetone), ethanol, and ethyl acetate are distilled and charged via the line 26 into a fourth distillation column H. The fourth distillation column in this case functions as a low-boiling component recovery column. A bottom liquid (water) is discharged via the line 31. Part of the overhead products is refluxed via the line 27 into the distillation column. The system also includes the condenser M-8 and the cooler M-10, the receiver R-2, the pumps N-13 and N-14, the reboiler O-3, and the low-boiling component tank K-7.

At the fourth distillation column (low-boiling component recovery column) H, low-boiling components such as acetone are recovered from the column top via the line 29, and a liquid mixture of ethanol, ethyl acetate (by-product), and the azeotropic solvent (such as ethyl acetate) is recovered from the column bottom via the line 28. Part of the overhead products is refluxed via the line 30 into the distillation column. The system also includes the condenser M-9 and the cooler M-10, the receiver R-3, the pumps N-12, N-14, and N-15, the reboiler O-4, the low-boiling component tank K-7, and the recovered ethanol/ethyl acetate tank K-8.

The mixture of ethanol, ethyl acetate, and the azeotropic solvent from the line 28 may be further subjected to distillation and/or extraction for separation according to necessity.

In an embodiment, the method according to the third embodiment of the present invention may be performed in the following manner. Pressures are controlled upon operation so that the temperature of the overhead vapor of the second distillation column is hither than the bottom temperature (column bottom temperature) of at least one distillation column selected from the group consisting of the first distillation column, the third distillation column, and the fourth distillation column; and the overhead vapor of the second distillation column is used as a heat source for heating the at least one distillation column (distillation column having a bottom temperature lower than the temperature of the overhead vapor of the second distillation column) selected from the group consisting of the first distillation column, the third distillation column, and the fourth distillation column. For example, the overhead vapor of the second distillation column may be used as a heat source for heating one distillation column, or two or three distillation columns selected from the first distillation column, the third distillation column, and the fourth distillation column. This configuration significantly reduces the energy cost of the entire purification system.

In this embodiment, the overhead vapor temperature of the second distillation column is controlled to be higher than the bottom temperature of at least one of the first distillation column, the third distillation column, and the fourth distillation column. The control may be performed typically by operating the columns so that the column top pressure of the second distillation column is higher than the column top pressure of at least one of the first distillation column, the third distillation column, and the fourth distillation column. For example, the overhead vapor temperature of the second distillation column may be controlled to be higher than the bottom temperature(s) of the other column(s) typically by operating the second distillation column under pressure and operating the other distillation column(s) at normal atmospheric pressure; or by operating the second distillation column under pressure and operating the other distillation column(s) under reduced pressure; or by operating the second distillation column at normal atmospheric pressure and operating the other distillation column(s) under reduced pressure.

In these cases, the overhead vapor temperature t of the second distillation column is higher than the bottom temperature(s) tx of the other distillation column(s) by typically 1° C. to 100° C., and preferably 5° C. to 50° C.

Ethanol Conversion into Ethyl Acetate

As described above, the lower-phase distillate from the acetic acid recovery column contains, as dissolved, ethyl acetate in addition to by-products acetone, ethanol, and water. Thus, part of ethyl acetate is discharged from the acetic acid recovery column. This requires replenishment of ethyl acetate, or recovery of dissolved ethyl acetate from the lower-phase distillate and recycling of the recovered ethyl acetate to the acetic acid recovery column. The replenishment of ethyl acetate leads to higher cost due to the cost of ethyl acetate to be replenished. Independently, the recovery of ethyl acetate also leads to higher cost, because ethyl acetate undergoes azeotropy also with ethanol, and separation and recovery of ethyl acetate alone from the lower-phase distillate requires a complicated process.

To eliminate or minimize these problems, the method according to the fourth embodiment of the present invention is performed in the following manner. After acetaldehyde, unreacted acetic acid, and water are separated from the crude reaction liquid via distillation to leave a fraction containing ethanol, the ethanol-containing fraction is combined with acetic acid, and the ethanol is converted into ethyl acetate in the presence of an acidic catalyst so as to increase an ethyl acetate to ethanol ratio (weight ratio). A liquid after the conversion has an ethyl acetate to ethanol ratio (weight ratio) of preferably 1 or more, and more preferably 3 or more.

Examples of the ethanol-containing fraction include, but are not limited to, the mixture of ethanol and ethyl acetate from the column top of the fourth distillation column in the first process; and the liquid mixture of ethanol and ethyl acetate from the column bottom of the fourth distillation column in the second process.

The acidic catalyst may be either of a homogeneous catalyst and a solid catalyst, as long as being an acidic catalyst capable of esterifying ethanol with acetic acid. The homogeneous catalyst may be selected from, but not limited to, mineral acids such as sulfuric acid and phosphoric acid; and organic acids such as p-toluenesulfonic acid and methanesulfonic acid. The solid catalyst may be selected from, but not limited to, ion exchange resins and zeolite.

The reactor may have any of a complete mixing structure and a plug flow structure, alone or in combination. The reactor may be configured to separate part or the whole of the product water and/or ethyl acetate in the middle of the reaction so as to enhance the reaction. The reactor may be a fixed bed reactor which is packed with a solid catalyst; and/or a catalyst may be arranged in the distillation column to perform the esterification reaction and the product separation simultaneously. The acidic catalyst, when contained in the esterification reaction liquid, is separable from the liquid by a common procedure.

The esterification reaction may be performed at a reaction temperature of typically 30° C. to 150° C., and preferably 40° C. to 100° C. The reaction may be performed under reduced pressure, at normal atmospheric pressure, or under pressure (under a load) without limitation.

The ethyl acetate converted from ethanol may be used in the acetaldehyde production process typically as any of the absorbing liquid in the absorber, the liquid to be charged into the acetaldehyde product column, and the liquid to be returned to the acetic acid recovery column (as the liquid to be charged through the column top). The separated acidic catalyst may be recycled to the esterification reaction.

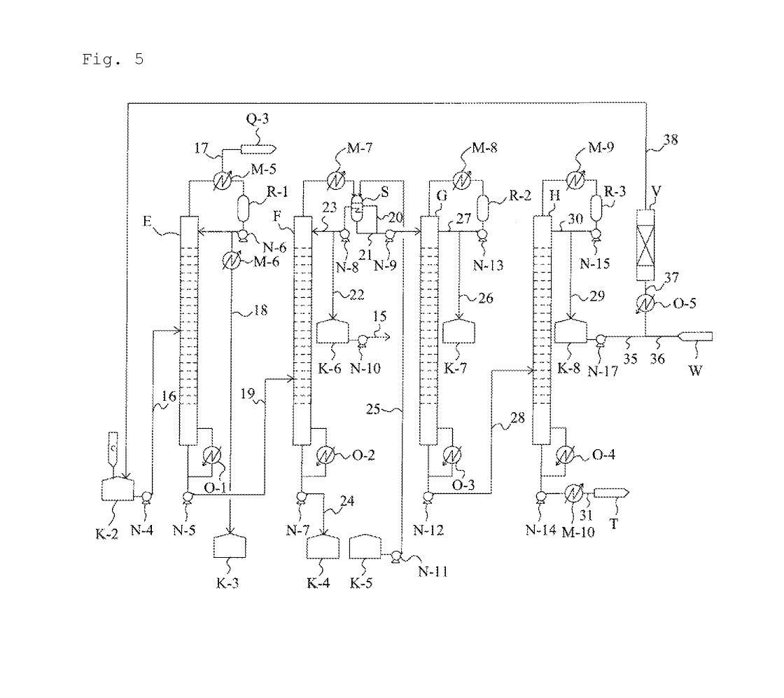

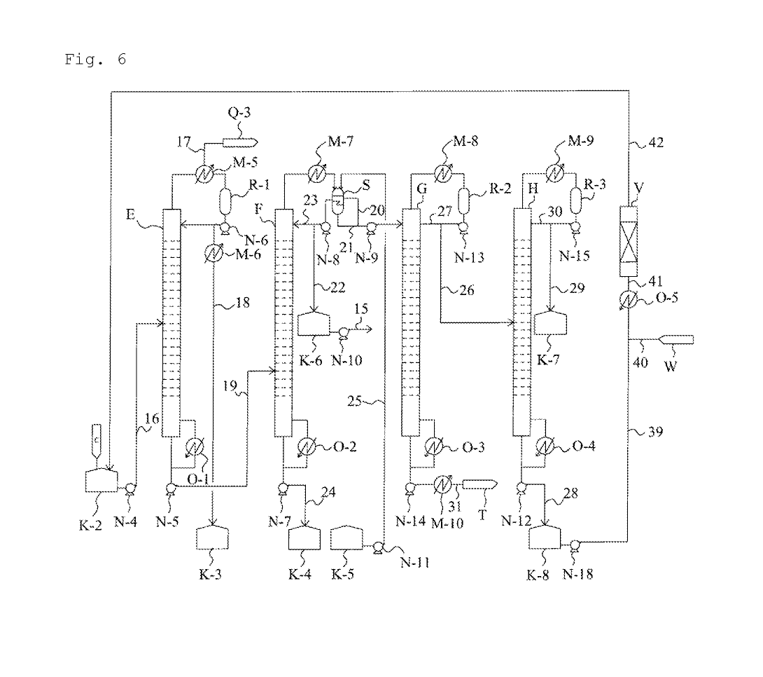

FIG. 5 is a schematic flow diagram illustrating a purification system (including the ethanol esterification step) according to the fourth embodiment of the present invention, where the purification system includes the first process; and FIG. 6 is a schematic flow diagram illustrating another purification system (including the ethanol esterification step) according to the fourth embodiment of the present invention, where the purification system includes the second process.

In the embodiment illustrated in FIG. 5, the crude reaction liquid is charged from the crude reaction liquid tank K-2 using the pump N-4 via the line 16 into the first distillation column (acetaldehyde product column) E. From the column top of the first distillation column (acetaldehyde product column) E, non-condensable gases are purged via the line 17, and acetaldehyde product is distilled via the line 18. A bottom liquid from the first distillation column (acetaldehyde product column) E is fed via the line 19 to the second distillation column (acetic acid recovery column) F. The system also includes the condensers M-5 and M-6, the receiver R-1, the pumps N-5 and N-6, the vent Q-3, the reboiler O-1, and the acetaldehyde product tank K-3.

At the second distillation column (acetic acid recovery column) F, an ethyl acetate-containing liquid is charged via the line 23 into the column top. Unreacted acetic acid is recovered as a bottom liquid via the line 24, stored in the recovered acetic acid tank K-4, and recycled to the reaction system. Acetone, ethanol, ethyl acetate, water, and the azeotropic solvent are distilled from the column top (as overheads) of the second distillation column (acetic acid recovery column) F, and separated with the decanter S into an upper-phase liquid and lower-phase water. Part of the upper-phase liquid (as needed) and the lower-phase water are charged respectively via the line 20 and the line 21 into the third distillation column (low-boiling component removal column) G. Ethyl acetate is fed from the ethyl acetate tank K-5 via the line 25 to the decanter S. Part of the upper-phase liquid is fed from the decanter S via the line 22, stored in the absorbing liquid tank K-6, and charged, via the line 15 and the line 11, also into the absorber C to absorb acetaldehyde, as described above. Another part of the upper-phase liquid from the decanter S is refluxed via the line 23 into the distillation column. The system also includes the condenser M-7, the pumps N-7, N-8, N-9, N-10, and N-11, and the reboiler O-2.

At the third distillation column (low-boiling component removal column) G, low-boiling components such as acetone are distilled from the column top via the line 26, and a bottom liquid from the column bottom is charged via the line 28 into the fourth distillation column (ethanol/ethyl acetate recovery column) H. Part of the overhead products is refluxed via the line 27 into the distillation column. The system also includes the condenser M-8, the receiver R-2, the pumps N-12 and N-13, the reboiler O-3, and the low-boiling component tank K-7.

At the fourth distillation column (ethanol/ethyl acetate recovery column) H, ethanol and ethyl acetate are recovered from the column top via the line 29, and a bottom liquid (water) is discharged via the line 31. Part of the overhead products is refluxed via the line 30 into the distillation column. The system also includes the condenser M-9 and the cooler M-10, the receiver R-3, the pumps N-14 and N-15, the reboiler O-4, and the recovered ethanol/ethyl acetate tank K-8.

Part or the whole of the ethanol/ethyl acetate mixture in the line 35 is combined with acetic acid fed via the line 36 so as to increase the ethyl acetate concentration, raised in temperature to an esterification reaction temperature with a heater O-5, and fed via a line 37 to an esterification reactor V in which the acidic catalyst is present, to esterify ethanol. The resulting liquid recycled via a line 38 typically to the acetaldehyde product column E. The residual ethanol/ethyl acetate mixture may be subjected to any of esterification reaction, distillation, and extraction for separation, according to necessity.

In the embodiment illustrated in FIG. 6, the crude reaction liquid is charged from the crude reaction liquid tank K-2 using the pump N-4 via the line 16 into the first distillation column (acetaldehyde product column) E. From the column top of the first distillation column (acetaldehyde product column) E, non-condensable gases are purged via the line 17, and acetaldehyde product is distilled via the line 18. A bottom liquid from the first distillation column (acetaldehyde product column) E is fed via the line 19 to the second distillation column (acetic acid recovery column) F. The system also includes the condensers M-5 and M-6, the receiver R-1, the pumps N-5 and N-6, the vent Q-3, the reboiler O-1, and the acetaldehyde product tank K-3.

At the second distillation column (acetic acid recovery column) F, an ethyl acetate-containing liquid is charged via the line 23 into the column top. Unreacted acetic acid is recovered as a bottom liquid via the line 24, stored in the recovered acetic acid tank K-4, and recycled to the reaction system. Acetone, ethanol, ethyl acetate, water, and the azeotropic solvent are distilled from the column top of the second distillation column (acetic acid recovery column) F, and separated with the decanter S into an upper-phase liquid and lower-phase water. Part of the upper-phase liquid (as needed) and the lower-phase water are charged respectively via the line 20 and the line 21 into a third distillation column G. The third distillation column in this case functions as a water separation column. An azeotropic solvent (such as ethyl acetate) is fed from the ethyl acetate tank K-5 via the line 25 to the decanter S. Part of the upper-phase liquid is fed from the decanter S via the line 22, stored in the absorbing liquid tank K-6, and charged, via the line 15 and the line 11, also into the absorber C to absorb acetaldehyde, as described above. Part of the upper-phase liquid is refluxed from the decanter S via the line 23 into the distillation column. The system also includes the condenser M-7, the pumps N-7, N-8, N-9, N-10, and N-11, and the reboiler O-2.

Low-boiling components (such as acetone), ethanol, and ethyl acetate are distilled from the column top of the third distillation column (water separation column) G and charged via the line 26 into a fourth distillation column H. The fourth distillation column in this case functions as a low-boiling component recovery column. A bottom liquid (water) is discharged via the line 31. Part of the overhead products is refluxed via the line 27 into the distillation column. The system also includes the condenser M-8 and the cooler M-10, the receiver R-2, the pumps N-13 and N-14, and the reboiler O-3.

At the fourth distillation column (low-boiling component recovery column) H, low-boiling components such as acetone are recovered from the column top via the line 29, and a liquid mixture of ethanol, ethyl acetate (by-product), and the azeotropic solvent (such as ethyl acetate) is recovered from the column bottom via the line 28. Part of the overhead products is refluxed via the line 30 into the distillation column. The system also includes the condenser M-9, the receiver R-3, the pumps N-12 and N-15, the reboiler O-4, the low-boiling component tank K-7, and the recovered ethanol/ethyl acetate tank K-8.

Part or the whole of the ethanol/ethyl acetate mixture in a line 39 is combined with acetic acid fed via a line 40 to increase ethyl acetate concentration, raised in temperature to an esterification reaction temperature with the heater O-5, and fed via a line 41 to the esterification reactor V in which the acidic catalyst is present, to esterify ethanol. The resulting liquid is recycled via a line 42 typically to the acetaldehyde product column E. The residual ethanol/ethyl acetate mixture may be further subjected to any of esterification reaction, distillation, and extraction for the separation, according to necessity.

Reaction System 2 (Reaction of Ethanol with Acetic Acid)

As described above, ethanol and ethyl acetate undergo azeotropy with each other. This requires a complicated process so as to separate a by-produced mixture of ethanol and ethyl acetate into ethanol and ethyl acetate and leads to higher costs for ethanol and ethyl acetate that are obtained as valuable substances.

To eliminate or minimize these problems, the method according to the fifth embodiment of the present invention is performed as follows. After acetaldehyde, unreacted acetic acid, and water are separated from the crude reaction liquid via distillation to leave a mixture of ethanol and ethyl acetate, part or the whole of the mixture is combined with acetic acid, and the ethanol is converted into ethyl acetate in the presence of an acidic catalyst. Exemplary processes for converting ethanol into ethyl acetate are found in GB 710,803 and SU (former Soviet Union) 857,109.

Examples of the mixture of ethanol and ethyl acetate include, but are not limited to, the mixture of ethanol and ethyl acetate from the fourth distillation column top in the first process; the liquid mixture of ethanol and ethyl acetate from the fourth distillation column bottom in the second process; and a mixture of ethanol and ethyl acetate further containing low-boiling components, from the third distillation column top.

From the reaction liquid after the esterification reaction, unreacted starting material(s) may be recovered and recycled using a common separation/purification procedure for ethyl acetate reaction liquids. This gives product ethyl acetate.

The acidic catalyst may be any of a homogeneous catalyst and a solid catalyst, as long as being an acidic catalyst capable of esterifying ethanol with acetic acid. The homogeneous catalyst may be selected typically, but not limitatively, from mineral acids such as sulfuric acid and phosphoric acid; and organic acids such as p-toluenesulfonic acid and methanesulfonic acid. The solid catalyst may be selected typically, but not limitatively, from ion exchange resins and zeolite.

The reactor may have any of a complete mixing structure and a plug flow structure, alone or in combination. The reactor may be configured to separate part or the whole of the product water and/or ethyl acetate in the middle of the reaction so as to enhance the reaction. The reactor may be a fixed bed reactor packed with a solid catalyst; and/or a catalyst may be arranged in the distillation column to perform the esterification reaction and the product separation simultaneously. The acidic catalyst, when contained in the esterification reaction liquid, may be separated from the liquid by a common procedure.

The esterification reaction may be performed at a reaction temperature of typically 30° C. to 150° C., and preferably 40° C. to 100° C. The reaction may be performed under reduced pressure, at normal atmospheric pressure, or under pressure (under a load) without limitation.

From the reaction liquid after the esterification reaction, unreacted starting material(s) may be recovered and recycled using a common separation/purification procedure for ethyl acetate reaction liquids. This gives product ethyl acetate.

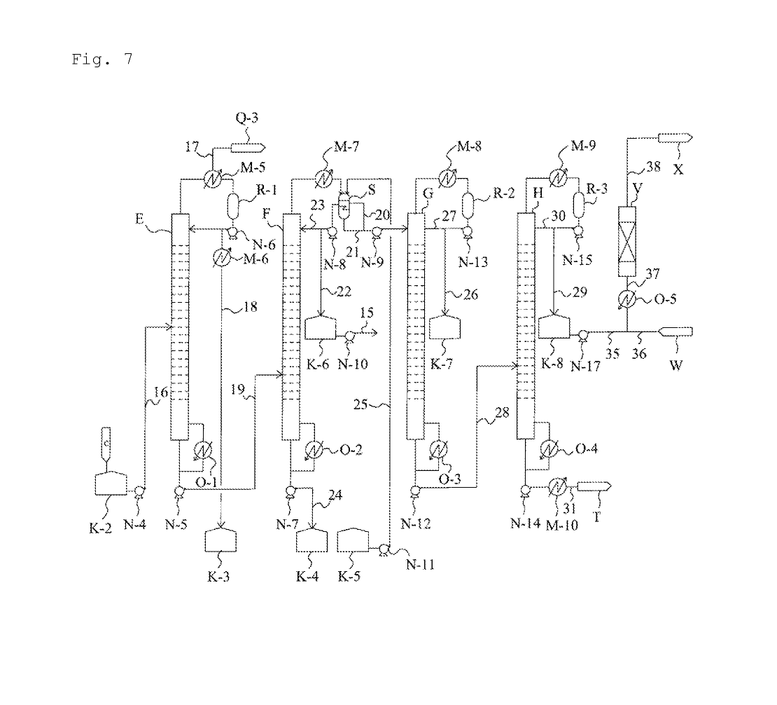

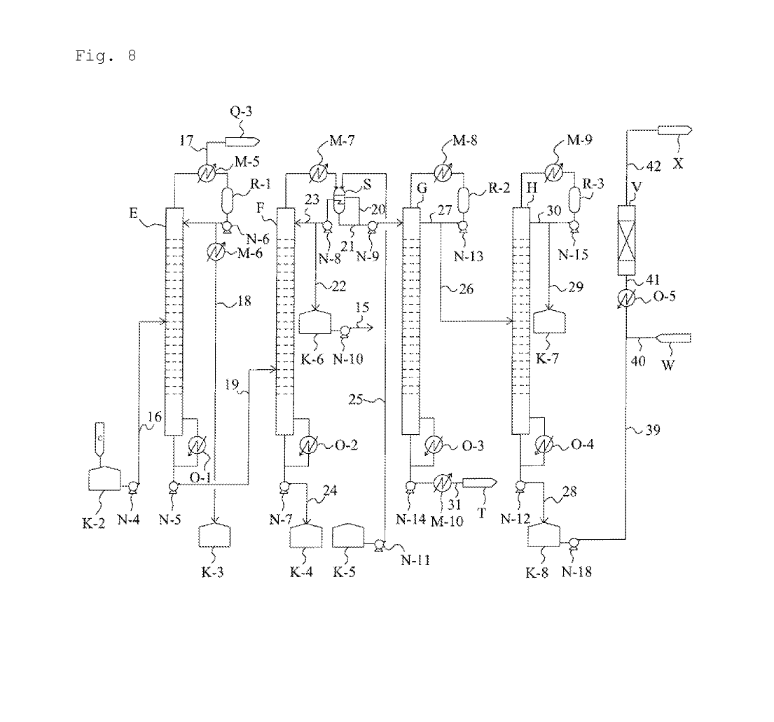

FIG. 7 is a schematic flow diagram illustrating a purification system (including the reaction system 2) according to the fifth embodiment of the present invention, where the purification system includes the first process; and FIG. 8 is a schematic flow diagram illustrating another purification system (including the reaction system 2) according to the fifth embodiment of the present invention, where the purification system includes the second process.

In the embodiment illustrated in FIG. 7, the crude reaction liquid is charged from the crude reaction liquid tank K-2 using the pump N-4 via the line 16 into the first distillation column (acetaldehyde product column) E. From the column top of the first distillation column (acetaldehyde product column) E, non-condensable gases are purged via the line 17, and acetaldehyde product is distilled via the line 18. A bottom liquid from the first distillation column (acetaldehyde product column) E is fed via the line 19 to the second distillation column (acetic acid recovery column) F. The system also includes the condensers M-5 and M-6, the receiver R-1, the pumps N-5 and N-6, the vent Q-3, the reboiler O-1, and the acetaldehyde product tank K-3.

At the second distillation column (acetic acid recovery column) F, an ethyl acetate-containing liquid is charged via the line 23 into the column top. Unreacted acetic acid is recovered as a bottom liquid via the line 24, stored in the recovered acetic acid tank K-4, and recycled to the reaction system. Acetone, ethanol, ethyl acetate, and water are distilled from the column top of the second distillation column (acetic acid recovery column) F, and separated with the decanter S into an upper-phase liquid and lower-phase water. Part of the upper-phase liquid (as needed) and the lower-phase water are charged respectively via the line 20 and the line 21 into a third distillation column (low-boiling component removal column) G. Ethyl acetate is fed from the ethyl acetate tank K-5 via the line 25 to the decanter S. Part of the upper-phase liquid is fed from the decanter S via the line 22, stored in the absorbing liquid tank K-6, and charged, via the line 15 and the line 11, also into the absorber C to absorb acetaldehyde, as described above. Another part of the upper-phase liquid is refluxed from the decanter S via the line 23 into the distillation column. The system also includes the condenser M-7, the pumps N-7, N-8, N-9, N-10, and N-11, and the reboiler O-2.

At the third distillation column (low-boiling component removal column) G, low-boiling components such as acetone are distilled from the column top via the line 26, and a bottom liquid from the column bottom is charged via the line 28 into a fourth distillation column (ethanol/ethyl acetate recovery column) H. Part of the overhead products is refluxed via the line 27 into the distillation column. The system also includes the condenser M-8, the receiver R-2, the pumps N-12 and N-13, the reboiler O-3, and the low-boiling component tank K-7.

At the fourth distillation column (ethanol/ethyl acetate recovery column) H, a mixture of ethanol and ethyl acetate is recovered from the column top via the line 29, and a bottom liquid (water) is discharged via the line 31. Part of the overhead products is refluxed via the line 30 into the distillation column. The system also includes the condenser M-9 and the cooler M-10, the receiver R-3, the pumps N-14 and N-15, the reboiler O-4, and the recovered ethanol/ethyl acetate tank K-8.

Part or the whole of the ethanol/ethyl acetate mixture is combined with acetic acid fed via the line 36, raised in temperature to an esterification reaction temperature with the heater O-5, fed via the line 37 to the esterification reactor V in which the acidic catalyst is present, to esterify ethanol. After the ethanol esterification, the resulting mixture is fed via the line 38 to an ethyl acetate purification process X. From the mixture (ethyl acetate reaction liquid), unreacted starting material(s) is recovered using a common separation/purification procedure for ethyl acetate reaction liquids. This gives product ethyl acetate.

In the embodiment illustrated in FIG. 8, the crude reaction liquid is charged from the crude reaction liquid tank K-2 using the pump N-4 via the line 16 into the first distillation column (acetaldehyde product column) E. From the column top of the first distillation column (acetaldehyde product column) E, non-condensable gases are purged via the line 17, and acetaldehyde product is distilled via the line 18. A bottom liquid from the first distillation column (acetaldehyde product column) E is fed via the line 19 to the second distillation column (acetic acid recovery column) F. The system also includes the condensers M-5 and M-6, the receiver R-1, the pumps N-5 and N-6, the vent Q-3, the reboiler O-1, and the acetaldehyde product tank K-3.

At the second distillation column (acetic acid recovery column) F, an ethyl acetate-containing liquid is charged via the line 23 into the column top. Unreacted acetic acid is recovered as a bottom liquid via the line 24, stored in the recovered acetic acid tank K-4, and recycled to the reaction system. Acetone, ethanol, ethyl acetate, and water are distilled from the column top of the second distillation column (acetic acid recovery column) F, and separated with the decanter S into an upper-phase liquid and lower-phase water. Part of the upper-phase liquid (as needed) and the lower-phase water are charged respectively via the line 20 and the line 21 into a third distillation column G. The third distillation column in this case functions as a water separation column. Ethyl acetate is fed from the ethyl acetate tank K-5 via the line 25 to the decanter S. Part of the upper-phase liquid is fed from the decanter S via the line 22, stored in the absorbing liquid tank K-6, and charged, via the line 15 and the line 11, also into the absorber C to absorb acetaldehyde, as described above. Another part of the upper-phase liquid is refluxed from the decanter S via the line 23 into the distillation column. The system also includes the condenser M-7, the pumps N-7, N-8, N-9, N-10, and N-11, and the reboiler O-2.

Low-boiling components (such as acetone), ethanol, and ethyl acetate are distilled from the column top of the third distillation column (water separation column) G and charged via the line 26 into a fourth distillation column H. The fourth distillation column in this case functions as a low-boiling component recovery column. A bottom liquid (water) is discharged via the line 31. Part of the overhead products is refluxed via the line 27 into the distillation column. The system also includes the condenser M-8 and the cooler M-10, the receiver R-2, the pumps N-13 and N-14, the reboiler O-3.

Low-boiling components such as acetone are recovered from the column top via the line 29, and a mixture of ethanol and ethyl acetate is recovered from the column bottom via the line 28. Part of the overhead products is refluxed via the line 30 into the distillation column. The system also includes the condenser M-9, the receiver R-3, the pumps N-12 and N-15, the reboiler O-4, the low-boiling component tank K-7, and the recovered ethanol/ethyl acetate tank K-8.

Part or the whole of the ethanol/ethyl acetate mixture in the line 39 is combined with acetic acid fed via the line 40, raised in temperature to an esterification reaction temperature with the heater O-5, and fed via the line 41 to the esterification reactor V in which the acidic catalyst is present, to esterify ethanol. After the ethanol esterification, the resulting mixture (ethyl acetate reaction liquid) is fed via the line 42 to the ethyl acetate purification process X, in which unreacted starting material(s) is recovered and recycled using a common separation/purification procedure for ethyl acetate reaction liquids. This gives product ethyl acetate.

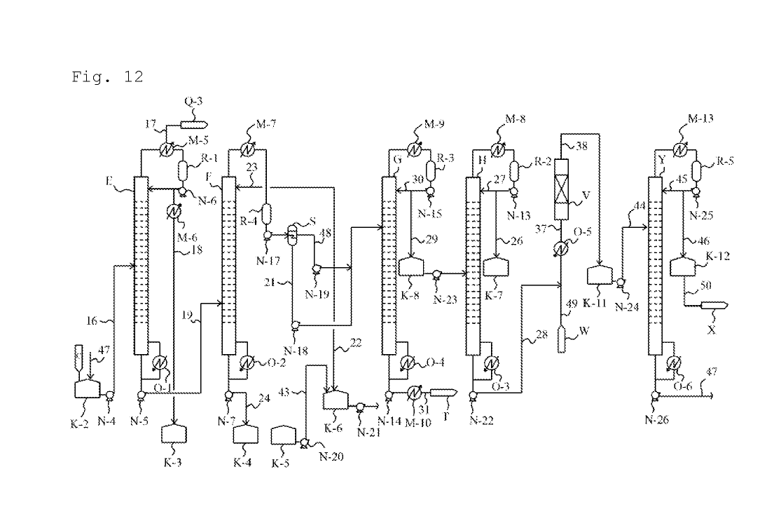

FIG. 11 is a schematic flow diagram illustrating a purification system according to the sixth embodiment of the present invention, where the purification system includes the first process; and FIG. 12 is a schematic flow diagram illustrating another purification system according to the sixth embodiment of the present invention, where the purification system includes the second process. Specifically, the method according to the sixth embodiment of the present invention includes the first process (1) or the second process (2). The first process (1) includes the steps of separating a low-boiling component from the residual liquid after the unreacted acetic acid separation, via distillation in a third distillation column, where the low-boiling component has a lower boiling point as compared with ethanol; and separating water and a mixture of ethanol and the azeotropic solvent from the residual liquid after the low-boiling component separation, via distillation in a fourth distillation column. The second process (2) includes the steps of separating water from the residual liquid after the unreacted acetic acid separation, via distillation in a third distillation column; and separating a low-boiling component and a mixture of ethanol and an azeotropic solvent from the residual liquid after the water separation, via distillation in a fourth distillation column, where the low-boiling component has a lower boiling point as compared with ethanol. The azeotropic solvent for use herein is as with the above-mentioned azeotropic solvent.

In the embodiment illustrated in FIG. 11, the crude reaction liquid is charged from the crude reaction liquid tank K-2 using the pump N-4 via the line 16 into the first distillation column (acetaldehyde product column) E. From the column top of the first distillation column (acetaldehyde product column) E, non-condensable gases are purged via the line 17, and acetaldehyde product is distilled via the line 18. A bottom liquid from the first distillation column (acetaldehyde product column) E is fed via the line 19 to the second distillation column (acetic acid recovery column) F. The system also includes the condensers M-5 and M-6, the receiver R-1, pumps N-4, N-5, and N-6, the vent Q-3, the reboiler O-1, and the acetaldehyde product tank K-3.

Acetone, ethanol, ethyl acetate, water, and the azeotropic solvent are distilled from the column top of the second distillation column (acetic acid recovery column) F. The distillates are separated into an upper-phase liquid and lower-phase water with the decanter S. The lower-phase water and part of the upper-phase liquid are charged respectively via the line 21 and a line 48 into a third distillation column (low-boiling component removal column) G. At the second distillation column (acetic acid recovery column) F, part of the upper-phase liquid from the decanter S is charged via the line 23 into the column top. Unreacted acetic acid is recovered as a bottom liquid via the line 24, stored in the recovered acetic acid tank K-4, and recycled to the reaction system. Part of the upper-phase liquid is fed from the decanter S via the line 22, stored in the absorbing liquid tank K-6, and charged, via the line 15 and the line 11, also into the absorber C to absorb acetaldehyde, as described above. The system also includes the condenser M-7, a receiver R-4, pumps N-7, N-17, N-18, N-19, N-20, and N-21, and the reboiler O-2.

At the third distillation column (low-boiling component removal column in FIG. 11) G, low-boiling components such as acetone is distilled from the column top via the line 26, and a bottom liquid from the column bottom is charged via the line 28 into a fourth distillation column (ethanol recovery column) H. Part of the overhead products is refluxed via the line 27 into the distillation column. The system also includes the condenser M-8, the receiver R-2, pumps N-13 and N-22, the reboiler O-3, and the low-boiling component tank K-7.

At the fourth distillation column (ethanol recovery column in FIG. 11) H, ethanol, ethyl acetate (by-product), the azeotropic solvent (such as ethyl acetate) are recovered from the column top via the line 29, and a bottom liquid (water) from the column bottom is discharged via the line 31. Part of the overhead products is refluxed via the line 30 into the distillation column. The system also includes the condenser M-9 and the cooler M-10, the receiver R-3, the pumps N-14, N-15, and N-23, the reboiler O-4, and the recovered ethanol tank K-8.

The distillate from the ethanol recovery column via the line 29 is combined with acetic acid fed via a line 49, charged into the esterification reactor V packed with an acidic catalyst, and converted into ethyl acetate by an esterification reaction. The acidic catalyst is preferably a strong acid ion exchange resin. The resulting esterification reaction liquid is fed via the line 38, stored in an esterification reaction liquid tank K-11, and charged via a line 44 into a fifth distillation column (ethyl acetate separation column) Y. The system also includes a pump N-24 and a reboiler O-5.

Ethyl acetate is distilled as an overhead product from the column top of the fifth distillation column (ethyl acetate separation column) Y, and part of the overhead product is refluxed via a line 45 into the distillation column. The distilled ethyl acetate is stored, via a line 46, in an ethyl acetate tank K-12, and fed via a line 50 to the ethyl acetate purification process X, in which unreacted starting material(s) is recovered using a common separation/purification procedure for ethyl acetate reaction liquids. This gives product ethyl acetate. A bottom liquid from the column bottom is recycled via a line 47 typically to the crude reaction liquid tank K-2. The system also includes a condenser M-13, a receiver R-5, pumps N-25 and N-26, and a reboiler O-6.

In the embodiment illustrated in FIG. 12, the crude reaction liquid is charged from the crude reaction liquid tank K-2 using the pump N-4 via the line 16 into the first distillation column (acetaldehyde product column) E. From the column top of the first distillation column (acetaldehyde product column) E, non-condensable gases are purged via the line 17, and acetaldehyde product is distilled via the line 18. A bottom liquid from the first distillation column (acetaldehyde product column) E is fed via the line 19 to the second distillation column (acetic acid recovery column) F. The system also includes the condensers M-5 and M-6, the receiver R-1, the pumps N-4, N-5, and N-6, the vent Q-3, the reboiler O-1, and the acetaldehyde product tank K-3.

Acetone, ethanol, ethyl acetate, water, and the azeotropic solvent are distilled from the column top of the second distillation column (acetic acid recovery column) F. The distillates are separated with the decanter S into an upper-phase liquid and lower-phase water. The lower-phase water and part of the upper-phase liquid are charged respectively via the line 21 and the line 48 into a third distillation column (ethanol recovery column) G. At the second distillation column (acetic acid recovery column) F, the part of the upper-phase liquid after separation with the decanter S is charged via the line 23 into the column top. Unreacted acetic acid is recovered as a bottom liquid via the line 24, stored in the recovered acetic acid tank K-4, and recycled to the reaction system. Part of the upper-phase liquid is fed from the decanter S via the line 22, stored in the absorbing liquid tank K-6, and charged, via the line 15 and the line 11, also into the absorber C to absorb acetaldehyde, as described above. The system also includes the condenser M-7, the receiver R-4, the pumps N-7, N-17, N-18, N-19, N-20, and N-21, and the reboiler O-2.

At the third distillation column (ethanol recovery column in FIG. 12) G, ethanol, ethyl acetate (by-product), and the azeotropic solvent (such as ethyl acetate) are recovered from the column top via the line 29, and a bottom liquid (water) is discharged via the line 31. Part of the overhead products is refluxed via the line 30 into the distillation column. The system also includes the condenser M-9 and the cooler M-10, the receiver R-3, the pumps N-14, N-15, and N-23, the reboiler O-4, and the recovered ethanol tank K-8.

At the fourth distillation column (low-boiling component removal column in FIG. 12) H, low-boiling components such as acetone are distilled from the column top via the line 26, and a bottom liquid from the column bottom is fed via the line 28 to an esterification reaction step. Part of the overhead products is refluxed via the line 27 into the distillation column. The system also includes the condenser M-8, the receiver R-2, the pumps N-13 and N-22, the reboiler O-3, and the low-boiling component tank K-7.

The bottom liquid in the line 28 is combined with acetic acid fed via the line 49, charged into the esterification reactor V packed with an acidic catalyst, and converted into ethyl acetate by an esterification reaction. The acidic catalyst is preferably a strong acid ion exchange resin. The resulting esterification reaction liquid is stored, via the line 38, in the esterification reaction liquid tank K-11, and charged via the line 44 into the fifth distillation column (ethyl acetate separation column) Y. The system also includes the pump N-24 and the reboiler O-5.

At the fifth distillation column (ethyl acetate separation column) Y, ethyl acetate is distilled from the column top, and part of the overhead product is refluxed via the line 45 into the distillation column. The distilled ethyl acetate is stored, via the line 46, in the ethyl acetate tank K-12, and fed via the line 50 to the ethyl acetate purification process X, in which unreacted starting material(s) is recovered using a common separation/purification procedure for ethyl acetate reaction liquids. This gives product ethyl acetate. A bottom liquid from the column bottom is recycled via the line 47 typically to the crude reaction liquid tank K-2. The system also includes the condenser M-13, the receiver R-5, the pumps N-25 and N-26, and the reboiler O-6.

With the method according to the sixth embodiment of the present invention as illustrated in FIG. 11 and FIG. 12, part of ethanol is converted into ethyl acetate and separated. This allows inexpensive and simple recycling typically of the azeotropic solvent. The process of converting part of ethanol into ethyl acetate, separating the converted ethyl acetate, and recycling the azeotropic solvent is effective particularly when the azeotropic solvent is an ester having a boiling point of 100° C. to 118° C. This is because it is difficult to separate ethanol from the azeotropic solvent of this type.

In an embodiment, the method according to the sixth embodiment of the present invention as illustrated in FIG. 11 and FIG. 12 may be performed in the following manner. Pressures are controlled upon operation so that the temperature of the overhead vapor of the second distillation column is higher than the bottom temperature (column bottom temperature) of at least one distillation column selected from the group consisting of the first distillation column, the third distillation column, the fourth distillation column, and the fifth distillation column; and the overhead vapor of the second distillation column is used as a heat source for heating the at least one distillation column (distillation column having a bottom temperature lower than the overhead vapor temperature of the second distillation column) selected from the group consisting of the first distillation column, the third distillation column, the fourth distillation column, and the fifth distillation column. For example, the overhead vapor of the second distillation column may be used as a heat source for heating one, or two, three, or four distillation columns selected from the first distillation column, the third distillation column, the fourth distillation column, and the fifth distillation column. The configuration as above contributes to significant reduction in energy cost of the entire purification system.

The overhead vapor temperature of the second distillation column is controlled to be higher than the bottom temperature of at least one of the first distillation column, the third distillation column, the fourth distillation column, and the fifth distillation column. The control may be performed typically by operating the columns so that the column top pressure of the second distillation column is higher than the column top pressure of at least one of the first distillation column, the third distillation column, the fourth distillation column, and the fifth distillation column. For example, the overhead vapor temperature of the second distillation column may be controlled to be higher than the bottom temperature(s) of the other column(s) typically by operating the second distillation column under pressure and operating the other distillation column(s) at normal atmospheric pressure; or by operating the second distillation column under pressure and operating the other distillation column(s) under reduced pressure; or by operating the second distillation column at normal atmospheric pressure and operating the other distillation column(s) under reduced pressure.

In these cases, the overhead vapor temperature t of the second distillation column may be higher than the bottom temperature(s) tx of the other distillation column(s) by typically 1° C. to 100° C., and preferably 5° C. to 50° C.

### EXAMPLES

The present invention will be illustrated in further detail with reference to several examples below. It should be noted, however, that the examples are by no means intended to limit the scope of the present invention.

Examples 1 and 2 and Comparative Examples 1 and 2 relate to the first embodiment of the present invention. Example 3 and Comparative Example 3 relate to the second embodiment of the present invention. Examples 4 and 5 relate to the third embodiment of the present invention. Example 6 relates to the fourth embodiment of the present invention. Example 7 relates to the fifth embodiment of the present invention. Example 8 relates to the sixth embodiment of the present invention.

### Example 1

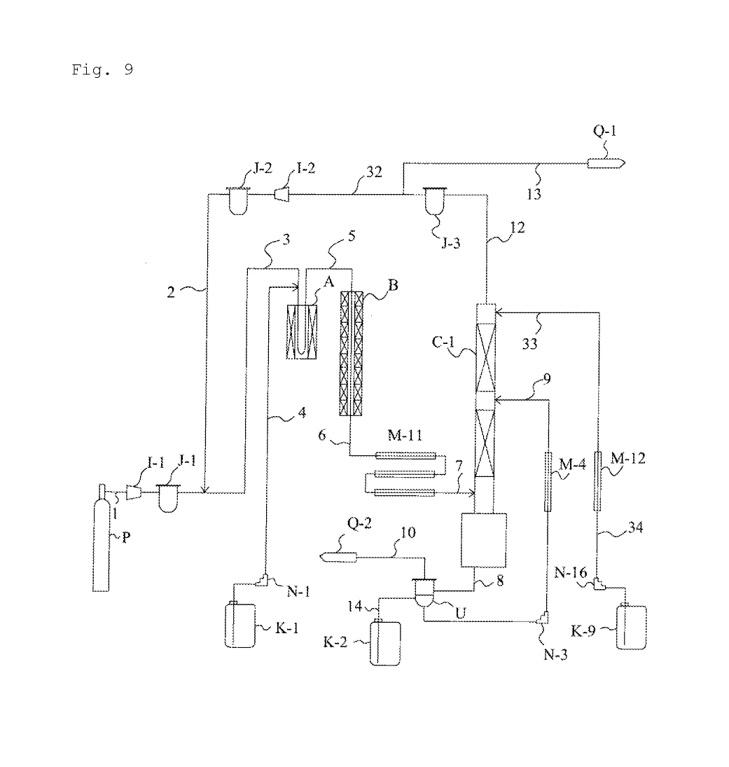

An acetic acid hydrogenation was performed using equipment illustrated in FIG. 9.

A gas from the column top of an after-mentioned absorber (scrubber) C-1, passing sequentially through a line 12 and a line 32 at a rate of 1,926 NL/hr, was compressed with a compressor I-2 and recycled via a line 2. Hydrogen was fed from a hydrogen cylinder P via a line 1 at a rate of 74 NL/hr, compressed with a compressor I-1 so as to keep the inlet pressure of an evaporator A constant at 1.7 MPa (gauge pressure), merged with the recycled gas, and charged via a line 3 into the evaporator A. The equipment also included buffer tanks J-1, J-2, and J-3.

Acetic acid was fed at a rate of 680 g/hr from an acetic acid tank K-1 via a line 4 into the evaporator (evaporator equipped with an electric heater) A, and, together with the hydrogen from the line 3, raised in temperature up to 300° C. to give a gaseous mixture of hydrogen and acetic acid. The gaseous mixture was charged into a reactor (reactor equipped with an electric heater) B. The reactor B was packed with 157 ml of a catalyst and had an outer diameter of 43.0 mm. The catalyst included 40 parts by weight of palladium (Pd) metal supported on 100 parts by weight of Fe₂O₃. The evaporator A and the reactor B each had an internal pressure of 1.7 MPa (gauge pressure). The reaction was performed at a temperature of 300° C. The equipment also included a pump N-1.

A reaction gas flown out from the reactor B via a line 6 was cooled down to 30° C. with a condenser (cooler) M-11 and charged via a line 7 into a lower portion of the absorber (scrubber) C-1. The absorber C-1 had an outer diameter of 48.6 and was packed with 6-mm diameter porcelain Raschig rings up to a height of 1 m. The absorber (scrubber) C-1 had an internal pressure of 1.7 MPa (gauge pressure). The equipment also included a pump N-3 and a cooler M-4.

An absorbing liquid at 30° C. was charged at a rate of 1,000 g/hr via a line 33 into an upper portion of the absorber (scrubber) C-1. The absorbing liquid was a liquid having a composition corresponding to the upper-phase distillate in the line 15 from the acetic acid recovery column F in FIG. 3 and included 3.1% by weight of acetone, 12.4% by weight of ethanol, 73.0% by weight of ethyl acetate, and 11.5% by weight of water. The absorbing liquid was fed from an absorbing liquid tank K-9 using a pump N-16 via a line 34 through a cooler M-12.

A bottom liquid of the absorber (scrubber) C-1 was drawn out via a line 8 to a gas-liquid separator U at normal atmospheric pressure so as to keep the liquid level at the bottom of the absorber (scrubber) C-1 constant, and dissolved gases were stripped from the liquid. The stripped gases were separated and removed via a line 10. Part of the residual liquid after the gas stripping was charged (recycled) at 30° C. at a rate of 10 L/hr via a line 9 into an intermediate portion of the absorber (scrubber) C-1.

The remainder of the liquid after the gas stripping was drawn out as a crude reaction liquid via a line 14 and stored in a crude reaction liquid tank K-2. The crude reaction liquid had a composition including 7.2% by weight of acetaldehyde, 2.0% by weight of acetone, 8.0% by weight of ethanol, 44.0% by weight of ethyl acetate, 10.2% by weight of water, and 28.6% by weight of acetic acid. The crude reaction liquid was produced in an amount of 1,667 g/hr.

No gas purging from a column top gas line 12 of the absorber (scrubber) C-1 via a line 13 coupled to a vent Q-1 was performed. However, a gas in the line 32 to be recycled to the evaporator A had a stable composition including 0.6% by mole of carbon dioxide, 1.1% by mole of methane, 1.2% by mole of ethane and ethylene, 0.7% by mole of propane and propylene, 0.2% by mole of acetaldehyde, and 96.2% by mole of hydrogen.

### Comparative Example 1

An acetic acid hydrogenation was performed using the equipment illustrated in FIG. 9.

A gas from the column top of the absorber (scrubber) C-1, passing sequentially through the line 12 and the line 32 at a rate of 1,926 NL/hr, was compressed with the compressor I-2 and recycled via the line 2. Separately, hydrogen was fed from the hydrogen cylinder P via the line 1 at a rate of 74 NL/hr, compressed with the compressor I-1, merged with the recycled gas, and charged via the line 3 into the evaporator A so that the evaporator A inlet pressure became constant at 1.7 MPa (gauge pressure). The equipment also included the buffer tanks J-1, J-2, and J-3.

Acetic acid was fed at a rate of 680 g/hr from the acetic acid tank K-1 via the line 4 into the evaporator (evaporator equipped with an electric heater) A and, together with the hydrogen fed from the line 3, raised in temperature up to 300° C. to give a gaseous mixture of hydrogen and acetic acid. The gaseous mixture was charged into the reactor (reactor equipped with an electric heater) B. The reactor B was packed with 157 ml of a catalyst and had an outer diameter of 43.0 mm. The catalyst included 40 parts by weight of palladium (Pd) metal supported on 100 parts by weight of Fe₂O₃. The evaporator A and the reactor B each had an internal pressure of 1.7 MPa (gauge pressure). The reaction was performed at a temperature of 300° C. The equipment also included the pump N-1.

A reaction gas flown out from the reactor B via the line 6 was cooled down to 30° C. with the condenser (cooler) M-11 and charged via the line 7 into a lower portion of the absorber (scrubber) C-1. The absorber C-1 had an outer diameter of 48.6 and was packed with 6-mm diameter porcelain Raschig rings up to a height of 1 m. The absorber (scrubber) C-1 had an internal pressure of 1.7 MPa (gauge pressure). The equipment also included the pump N-3 and the cooler M-4.

An absorbing liquid at 30° C. was charged at a rate of 1,000 g/hr via the line 33 into an upper portion of the absorber (scrubber) C-1. The absorbing liquid had a composition corresponding to the upper-phase distillate in the line 15 from the acetic acid recovery column F in FIG. 3 and included 3.1% by weight of acetone, 12.4% by weight of ethanol, 73.0% by weight of ethyl acetate, and 11.5% by weight of water. The absorbing liquid was fed from the absorbing liquid tank K-9 using the pump N-16 via the line 34 through the cooler M-12.

A bottom liquid of the absorber (scrubber) C-1 was drawn out via the line 8 to the gas-liquid separator U at normal atmospheric pressure so as to keep the liquid level at the bottom of the absorber (scrubber) C-1 constant, and dissolved gases were stripped from the liquid. The stripped gases were separated and removed via the line 10. In this comparative example, the whole quantity of the residual liquid after the gas stripping was drawn out via the line 14 into the crude reaction liquid tank K-2, without recycling of the bottom liquid from the absorber (scrubber) C-1 via the line 8 to the absorber (scrubber) C-1.

As the operation was continued, carbon dioxide and methane concentrations in the gas in the line 32 to be recycled to the evaporator A gradually increased, and the charge amount of hydrogen decreased. Accordingly, gas purging at a rate of 41 NL/hr was performed via the line 13 to the vent Q-1, and the amount of hydrogen to be charged into the evaporator A was increased by 38 NL/hr up to 112 NL/hr. As a result, the composition of the gas in the line 32 was stabilized at levels of 1.5% by mole of carbon dioxide, 1.5% by mole of methane, 2.4% by mole of ethane and ethylene, 1.9% by mole of propane and propylene, and 92.7% by mole of hydrogen.

A crude reaction liquid was obtained at a rate of 1,667 g/hr from a line 14, where the line 14 was directly coupled to the bottom liquid line 8 of the absorber (scrubber) C-1. The crude reaction liquid included 7.2% by weight of acetaldehyde, 2.0% by weight of acetone, 8.0% by weight of ethanol, 44.0% by weight of ethyl acetate, 10.2% by weight of water, and 28.6% by weight of acetic acid.

### Example 2

An acetic acid hydrogenation was performed using the equipment illustrated in FIG. 9.

A gas from the column top of the absorber (scrubber) C-1, passing sequentially through the line 12 and the line 32 at a rate of 1,923 NL/hr, was compressed with the compressor I-2 and recycled via the line 2. Hydrogen was fed from the hydrogen cylinder P via the line 1 at a rate of 77 NL/hr, compressed with the compressor I-1, merged with the recycled gas, and charged via the line 3 into the evaporator A so that the evaporator A inlet pressure became constant at 1.7 MPa (gauge pressure). The equipment also included the buffer tanks J-1, J-2, and J-3.

Acetic acid was fed at a rate of 677 g/hr from the acetic acid tank K-1 via the line 4 into the evaporator (evaporator equipped with an electric heater) A and, together with the hydrogen from the line 3, raised in temperature up to 300° C. to give a gaseous mixture of hydrogen and acetic acid. The gaseous mixture was charged into the reactor B. The reactor B was a reactor equipped with an electric heater, was packed with 157 ml of a catalyst, and had an outer diameter of 43.0 mm. The catalyst included 40 parts by weight of palladium (Pd) metal supported on 100 parts by weight of Fe₂O₃. The evaporator A and the reactor B each had an internal pressure of 1.7 MPa (gauge pressure). The reaction was performed at a temperature of 300° C. The equipment also included the pump N-1.

A reaction gas flown out from the reactor B via the line 6 was cooled down to 30° C. with the condenser (cooler) M-11 and charged via the line 7 into a lower portion of the absorber (scrubber) C-1. The absorber C-1 was packed with 6-mm diameter porcelain Raschig rings up to a height of 1 m and had an outer diameter of 48.6. The absorber (scrubber) C-1 had an internal pressure of 1.7 MPa (gauge pressure). The equipment also included the pump N-3 and the cooler M-4.

An absorbing liquid at 30° C. was charged at a rate of 1,000 g/hr via the line 33 into an upper portion of the absorber (scrubber) C-1. The absorbing liquid was a liquid having a composition corresponding to the bottom liquid in the line 19 from the acetaldehyde product column E in FIG. 3 and included 0.4% by weight of acetone, 1.8% by weight of ethanol, 0.8% by weight of ethyl acetate, 10.2% by weight of water, and 86.8% by weight of acetic acid. The absorbing liquid was fed from the absorbing liquid tank K-9 using the pump N-16 via the line 34 through the cooler M-12.

A bottom liquid of the absorber (scrubber) C-1 was drawn out via the line 8 to the gas-liquid separator U at normal atmospheric pressure so as to keep the liquid level at the bottom of the absorber (scrubber) C-1 constant, and dissolved gases were stripped from the liquid. The stripped gases were separated and removed via the line 10. Part of the residual liquid after the gas stripping was charged (recycled) at 30° C. at a rate of 26 L/hr via the line 9 into an intermediate portion of the absorber (scrubber) C.

The remainder of the liquid after the gas stripping was drawn out as a crude reaction liquid via the line 14 and stored in the crude reaction liquid tank K-2. The crude reaction liquid had a composition including 7.2% by weight of acetaldehyde, 0.4% by weight of acetone, 1.7% by weight of ethanol, 0.7% by weight of ethyl acetate, 9.4% by weight of water, and 80.6% by weight of acetic acid. The crude reaction liquid was produced in an amount of 1,659 g/hr.

No gas purging from the column top gas line 12 of the absorber (scrubber) C-1 via the line 13 to the vent Q-1 was performed. However, the gas in the line 32 to be recycled to the evaporator A had a stable composition including 1.2% by mole of carbon dioxide, 1.1% by mole of methane, 1.2% by mole of ethane and ethylene, 0.7% by mole of propane and propylene, 0.2% by mole of acetaldehyde, and 95.6% by mole of hydrogen.

### Comparative Example 2

An acetic acid hydrogenation was performed using the equipment illustrated in FIG. 9.

A gas from the column top of the absorber (scrubber) C-1, passing sequentially through the line 12 and the line 32 at a rate of 1,923 NL/hr, was compressed with the compressor I-2 and recycled via the line 2. Hydrogen was fed from the hydrogen cylinder P via the line 1 at a rate of 77 NL/hr, compressed with the compressor I-1, merged with the recycled gas, and charged via the line 3 into the evaporator A so that the evaporator A inlet pressure became constant at 1.7 MPa (gauge pressure). The equipment also included the buffer tanks J-1, J-2, and J-3.

Acetic acid was fed at a rate of 677 g/hr from the acetic acid tank K-1 via the line 4 into the evaporator (evaporator equipped with an electric heater) A and, together with the hydrogen from the line 3, raised in temperature up to 300° C. to give a gaseous mixture of hydrogen and acetic acid. The gaseous mixture was charged into the reactor B. The reactor B was a reactor equipped with an electric heater, was packed with 157 ml of a catalyst, and had an outer diameter of 43.0 mm. The catalyst included 40 parts by weight of palladium (Pd) metal supported on 100 parts by weight of Fe₂O₃. The evaporator A and the reactor B each had an internal pressure of 1.7 MPa (gauge pressure). The reaction was performed at a temperature of 300° C. The equipment also included the pump N-1.

A reaction gas flown out from the reactor B via the line 6 was cooled down to 30° C. with the condenser (cooler) M-11 and charged via the line 7 into a lower portion of the absorber (scrubber) C-1. The absorber C-1 was packed with 6-mm diameter porcelain Raschig rings up to a height of 1 m and had an outer diameter of 48.6. The absorber (scrubber) C-1 had an internal pressure of 1.7 MPa (gauge pressure). The equipment also included the pump N-3 and the cooler M-4.

An absorbing liquid at 30° C. was charged at a rate of 1,000 g/hr via the line 33 into an upper portion of the absorber (scrubber) C-1. The absorbing liquid was a liquid having a composition corresponding to the bottom liquid in the line 19 from the acetaldehyde product column E in FIG. 3, and included 0.4% by weight of acetone, 1.8% by weight of ethanol, 0.8% by weight of ethyl acetate, 10.2% by weight of water, and 86.8% by weight of acetic acid. The absorbing liquid was fed from the absorbing liquid tank K-9 using the pump N-16 via the line 34 through the cooler M-12.

A bottom liquid of the absorber (scrubber) C-1 was drawn out via the line 8 to the gas-liquid separator U at normal atmospheric pressure so as to keep the liquid level at the bottom of the absorber (scrubber) C-1 constant, and dissolved gases were stripped from the liquid. The stripped gases were separated and removed via the line 10. In this comparative example, the whole quantity of the residual liquid after the gas stripping was drawn out via the line 14 to the crude reaction liquid tank K-2 without recycling of the bottom liquid from the absorber (scrubber) C-1 via the line 8 to the absorber (scrubber) C-1.

As the operation was continued, carbon dioxide and methane concentrations in the gas in the line 32 to be recycled to the evaporator A gradually increased, and the charge amount of hydrogen decreased. Accordingly, gas purging at a rate of 41 NL/hr was performed via the line 13 to the vent Q-1, and the amount of hydrogen to be charged into the evaporator A was increased by 38 NL/hr up to 115 NL/hr. As a result, the composition of the gas in the line 32 was stabilized at levels of 1.5% by mole of carbon dioxide, 1.5% by mole of methane, 2.4% by mole of ethane and ethylene, 1.9% by mole of propane and propylene, and 92.7% by mole of hydrogen.

A crude reaction liquid was obtained at a rate of 1,659 g/hr from a line 14, where the line 14 was directly coupled to the bottom liquid line 8 of the absorber (scrubber) C-1. The crude reaction liquid included 7.2% by weight of acetaldehyde, 0.4% by weight of acetone, 1.7% by weight of ethanol, 0.7% by weight of ethyl acetate, 9.4% by weight of water, and 80.6% by weight of acetic acid.

### Example 3

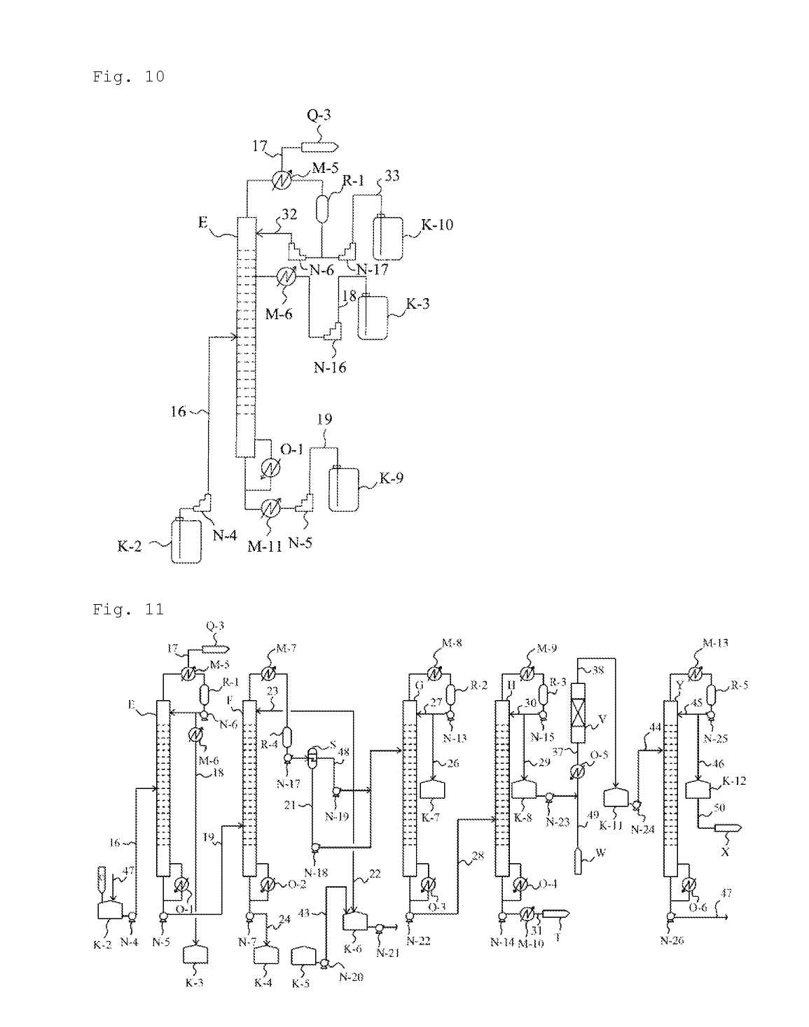

Acetaldehyde product was separated from a crude reaction liquid at normal atmospheric pressure by side-cutting using a glass distillation column E illustrated in FIG. 10. The distillation column E herein included 30 theoretical trays, had a diameter of 40 mm, and was equipped with a vacuum jacket. The crude reaction liquid had been obtained via acetic acid hydrogenation.

Specifically, the crude reaction liquid from the acetic acid hydrogenation was continuously charged at a rate of 1,000 g/hr via a line 16 using a pump into the twentieth theoretical tray from the column top of the distillation column E. The crude reaction liquid included 7.2% by weight of acetaldehyde, 2.0% by weight of acetone, 8.0% by weight of ethanol, 44.0% by weight of ethyl acetate, 10.2% by weight of water, and 28.6% by weight of acetic acid.

The heat medium temperature at the bottom was controlled so that the distillate amount be 300 ml/hr, and the whole quantity of the distillate was continuously refluxed using a pump N-6 via a line 32 to the column top.

A liquid in an uppermost tray was cooled down to 15° C. and continuously side-cut at a rate of 72 g/hr via a line 18 using a pump N-16.

A bottom liquid was cooled down to 30° C. and continuously drawn out using a pump N-5 via a line 19 at a rate of 928 g/hr so as to keep the bottom liquid level constant.

The side-cut liquid in the line 18 was acetaldehyde containing 1.8% by weight of low-boiling components and having a purity of 98.2% by weight.

The bottom liquid in the line 19 included 0.1% by weight of acetaldehyde, 2.1% by weight of acetone, 8.7% by weight of ethanol, 47.3% by weight of ethyl acetate, 11.0% by weight of water, and 30.8% by weight of acetic acid.

### Comparative Example 3

Acetaldehyde product was separated at normal atmospheric pressure from a crude reaction liquid using the glass distillation column E illustrated in FIG. 10, where the crude reaction liquid was derived from the acetic acid hydrogenation. The distillation column E included 30 theoretical trays, had a diameter of 40 mm, and was equipped with a vacuum jacket.

Specifically, the crude reaction liquid from the acetic acid hydrogenation was continuously charged at a rate of 1,000 g/hr via the line 16 using a pump onto the twentieth theoretical tray from the column top of the distillation column E. The crude reaction liquid included 7.2% by weight of acetaldehyde, 2.0% by weight of acetone, 8.0% by weight of ethanol, 44.0% by weight of ethyl acetate, 10.2% by weight of water, and 28.6% by weight of acetic acid.

A distillate was continuously refluxed at a rate of 300 ml/hr using a pump through the line N-6 via the line 32 to the column top, and acetaldehyde product was continuously drawn out at a rate of 72 g/hr using the pump N-17 via the line 33.

The heat medium temperature at the bottom was controlled so as to keep the distillate receiver liquid level constant. Side-cutting via the line 18 was not performed.

A bottom liquid was cooled down to 30° C. and continuously drawn out using the pump N-5 via the line 19 at a rate of 928 g/hr so as to keep the bottom liquid level constant.

The distillate in the line 33 was acetaldehyde containing 3.5% by weight of low-boiling components and having a purity of 96.5% by weight.

The bottom liquid in the line 19 included 0.1% by weight of acetaldehyde, 2.1% by weight of acetone, 8.7% by weight of ethanol, 47.3% by weight of ethyl acetate, 11.0% by weight of water, and 30.8% by weight of acetic acid.

### Example 4

A crude reaction liquid obtained by the method according to Example 1 was purified via the flow illustrated in FIG. 3.

A first distillation column (acetaldehyde product column) E herein included a glass distillation column including 30 theoretical trays, having a diameter of 50 mm, and equipped with a vacuum jacket. Onto the twentieth theoretical tray from the column top of the first distillation column E, the crude reaction liquid from the acetic acid hydrogenation was charged via the line 16 and distilled at normal atmospheric pressure at a reflux ratio of 3. An overhead vapor had a temperature of 21° C., was cooled down to 10° C., and drawn out via the line 18 to give acetaldehyde product at a rate of 120 g/hr. A bottom liquid had a temperature of 79° C. and was continuously drawn out via the line 19 at a rate of 1,547 g/hr so as to keep the liquid level constant. The bottom liquid included 2.1% by weight of acetone, 8.7% by weight of ethanol, 47.5% by weight of ethyl acetate, 11.0% by weight of water, and 30.8% by weight of acetic acid.

The bottom liquid was charged onto the twentieth theoretical tray of a second distillation column (acetic acid recovery column) F, where the second distillation column herein included a metallic distillation column including 30 theoretical trays and having a diameter of 100 mm. In addition, an upper-phase liquid was charged at a rate of 1,500 g/hr via the line 23, and distillation was performed at 190 kPa gauge pressure, where the upper-phase liquid had been separated, with the decanter S, from a distillate of the second distillation column (acetic acid recovery column) F. An overhead vapor had a temperature of 103° C., was condensed into a distillate and cooled down to 20° C. with the condenser M-7, and separated into an upper-phase liquid and a lower-phase liquid with the decanter S. Of the upper-phase liquid, 1,500 g/hr were refluxed to the second distillation column (acetic acid recovery column) F as described above, and 1,000 g/hr were recycled as the absorbing liquid to the acetic acid hydrogenation process. The bottom liquid had a temperature of 157° C. and was continuously drawn out via the line 24 at a rate of 477 g/hr so as to keep the liquid level constant. The bottom liquid included 0.1% by weight of water and 99.9% by weight of acetic acid. The decanter lower-phase liquid obtained at 79 g/hr included 3.1% by weight of acetone, 13.8% by weight of ethanol, 13.0% by weight of ethyl acetate, and 70.1% by weight of water.

The lower-phase liquid was charged onto the tenth theoretical tray from the column top of a third distillation column (low-boiling component removal column) G and subjected to distillation at normal atmospheric pressure at a reflux ratio of 210. The third distillation column G herein included a glass distillation column including 30 theoretical trays, having a diameter of 40 mm, and equipped with a vacuum jacket. An overhead vapor had a temperature of 59° C. and gave a distillate at 3 g/hr. The distillate included 79.2% by weight of acetone, 3.6% by weight of ethanol, 15.0% by weight of ethyl acetate, and 2.2% by weight of water. A bottom liquid had a temperature of 73° C. and was continuously drawn out via the line 28 at a rate of 76 g/hr so as to keep the liquid level constant. The bottom liquid included 14.2% by weight of ethanol, 12.9% by weight of ethyl acetate, and 72.9% by weight of water.

A fourth distillation column (ethanol/ethyl acetate recovery column) H herein included a glass distillation column including 10 theoretical trays, having a diameter of 40 mm, and equipped with a vacuum jacket. The bottom liquid was charged onto the fifth theoretical tray from the column top of the fourth distillation column H and subjected to distillation at 40 kPa (absolute pressure) and at a reflux ratio of 1.1. An overhead vapor had a temperature of 49° C. and gave a distillate at 23 g/hr. The distillate included 47.1% by weight of ethanol, 42.9% by weight of ethyl acetate, and 10.0% by weight of water. A bottom liquid had a temperature of 78° C. and was continuously drawn out via the line 31 at a rate of 53 g/hr so as to keep the liquid level constant. The bottom liquid included 0.1% by weight of ethanol and 99.9% by weight of water.

The overhead temperatures and the bottom temperatures of the distillation columns are summarized in Table 1.

The overhead temperature of the second distillation column (acetic acid recovery column) F is higher than the bottom temperatures of the first distillation column (acetaldehyde product column) E, the third distillation column (low-boiling component removal column) G, and the fourth distillation column (ethanol/ethyl acetate recovery column) H. Accordingly, the overhead vapor from the second distillation column (acetic acid recovery column) F is usable for heating at least one distillation column selected from the group consisting of the first distillation column (acetaldehyde product column) E, the third distillation column (low-boiling component removal column) G, and the fourth distillation column (ethanol/ethyl acetate recovery column) H.

### Example 5

A crude reaction liquid obtained by the method according to Example 1 was purified via the flow illustrated in FIG. 3.

The crude reaction liquid from the acetic acid hydrogenation was purified by a procedure similar to Example 4, except for the following points. A second distillation column (acetic acid recovery column) F herein included a metallic distillation column including 30 theoretical trays and having a diameter of 100 mm. At the second distillation column (acetic acid recovery column) F, the bottom liquid from the first distillation column (acetaldehyde product column) E was charged onto the twentieth theoretical tray from the column top; an upper-phase liquid was charged at 1,500 g/hr via the line 23; and distillation was performed at normal atmospheric pressure, where the upper-phase liquid had been separated, with the decanter S, from the distillate of the second distillation column (acetic acid recovery column) F.

An overhead vapor of the second distillation column (acetic acid recovery column) F had a temperature of 70° C., was condensed into a distillate and cooled down to 40° C. with the condenser M-7, and separated into an upper-phase liquid and a lower-phase liquid with the decanter S. Of the upper-phase liquid, 2,000 g/hr were refluxed to the second distillation column (acetic acid recovery column) F as described above, and 1,000 g/hr were recycled as the absorbing liquid to the acetic acid hydrogenation process. A bottom liquid had a temperature of 121° C. and was continuously drawn out via the line 24 at a rate of 477 g/hr so as to keep the liquid level constant. The bottom liquid included 0.1% by weight of water and 99.9% by weight of acetic acid. The decanter lower-phase liquid obtained at 79 g/hr included 3.1% by weight of acetone, 13.8% by weight of ethanol, 13.0% by weight of ethyl acetate, and 70.1% by weight of water.

Table 1 shows the overhead temperature and the bottom temperature of the second distillation column (acetic acid recovery column) F when the second distillation column was operated at normal atmospheric pressure.

The method according to this example efficiently separates and recovers acetaldehyde product, unreacted acetic acid, ethanol and ethyl acetate, and low-boiling components such as acetone in a short process, as with Example 4.

However, the overhead vapor of the second distillation column (acetic acid recovery column) F is not usable for heating of the other distillation columns, because the overhead temperature of the second distillation column (acetic acid recovery column) F is lower than the bottom temperatures of the first distillation column (acetaldehyde product column) E, the third distillation column (low-boiling component removal column) G, and the fourth distillation column (ethanol/ethyl acetate recovery column) H.

TABLE 1

| | | Overhead | Bottom | |
|---|---|---|---|---|
| | | temper- | temper- | |
| | Operation | ature | ature | |
| | pressure | (° C.) | (° C.) | Remarks |
| First distillation | 0 kPa | 21 | 79 | Example |
| column | (gauge pressure) | | | 4 |
| Second distillation | 190 kPa | 103 | 157 | Example |
| column | (gauge pressure) | | | 4 |
| | 0 kPa | 70 | 121 | Example |
| | (gauge pressure) | | | 5 |
| Third distillation | 0 kPa | 59 | 73 | Example |
| column | (gauge pressure) | | | 4 |
| Fourth distillation | 40 kPa | 49 | 78 | Example |
| column | (absolute | | | 4 |
| | pressure) | | | |

### Example 6

A crude reaction liquid obtained by the method according to Example 1 was purified by the flow illustrated in FIG. 5.

A first distillation column (acetaldehyde product column) E herein included a glass distillation column including 30 theoretical trays, having a diameter of 50 mm, and equipped with a vacuum jacket. Onto the twentieth theoretical tray from the column top of the first distillation column E, the crude reaction liquid from the acetic acid hydrogenation was charged via the line 16, and subjected to distillation at normal atmospheric pressure at a reflux ratio of 3. An overhead vapor had a temperature of 21° C., was cooled down to 10° C., and drawn out via the line 18 to give acetaldehyde product at 120 g/hr. A bottom liquid had a temperature of 79° C. and was continuously drawn out via the line 19 at 1,547 g/hr so as to keep the liquid level constant. The bottom liquid included 2.1% by weight of acetone, 8.7% by weight of ethanol, 47.5% by weight of ethyl acetate, 11.0% by weight of water, and 30.8% by weight of acetic acid.

A second distillation column (acetic acid recovery column) F herein included a metallic distillation column including 30 theoretical trays and having a diameter of 100 mm. At the second distillation column F, the bottom liquid was charged onto the twentieth theoretical tray, an upper-phase liquid was charged at 1,500 g/hr via the line 23, and distillation was performed at 190 kPa gauge pressure, where the upper-phase liquid had been separated from the distillate of the second distillation column (acetic acid recovery column) F with the decanter S. An overhead vapor had a temperature of 103° C., was condensed into a distillate and cooled down to 20° C. with the condenser M-7, and separated into an upper-phase liquid and a lower-phase liquid with the decanter S. Of the upper-phase liquid, 1,500 g/hr were refluxed to the second distillation column (acetic acid recovery column) F as described above, and 1,000 g/hr were recycled as the absorbing liquid to the acetic acid hydrogenation process.

A bottom liquid had a temperature of 157° C. and was continuously drawn out via the line 24 at a rate of 477 g/hr so as to keep the liquid level constant. The bottom liquid included 0.1% by weight of water and 99.9% by weight of acetic acid. The decanter lower-phase liquid was obtained at 79 g/hr and included 3.1% by weight of acetone, 13.8% by weight of ethanol, 13.0% by weight of ethyl acetate, and 70.1% by weight of water.

A third distillation column (low-boiling component removal column) G herein included a glass distillation column including 30 theoretical trays, having a diameter of 40 mm, and equipped with a vacuum jacket. The lower-phase liquid was charged onto the tenth theoretical tray from the column top of the third distillation column G, and subjected to distillation at normal atmospheric pressure at a reflux ratio of 210. An overhead vapor had a temperature of 59° C. and gave a distillate at 3 g/hr. The distillate included 79.2% by weight of acetone, 3.6% by weight of ethanol, 15.0% by weight of ethyl acetate, and 2.2% by weight of water. A bottom liquid had a temperature of 73° C. and was continuously drawn out via the line 28 at a rate of 76 g/hr so as to keep the liquid level constant. The bottom liquid included 14.2% by weight of ethanol, 12.9% by weight of ethyl acetate, and 72.9% by weight of water.

A fourth distillation column (ethanol/ethyl acetate recovery column) H herein included a glass distillation column including 10 theoretical trays, having a diameter of 40 mm, and equipped with a vacuum jacket. The bottom liquid was charged onto the fifth theoretical tray from the column top of the fourth distillation column H, and subjected to distillation at 40 kPa (absolute pressure) and at a reflux ratio of 1.1. An overhead vapor had a temperature of 49° C. and gave a distillate at 23 g/hr. The distillate included 47.1% by weight of ethanol, 42.9% by weight of ethyl acetate, and 10.0% by weight of water. A bottom liquid had a temperature of 78° C. and was continuously drawn out via the line 31 at 53 g/hr so as to keep the liquid level constant. The bottom liquid included 0.1% by weight of ethanol and 99.9% by weight of water.

Acetic acid (245 parts by weight) was added to the distillate (100 parts by weight) of the ethanol/ethyl acetate recovery column including 47.1% by weight of ethanol, 42.9% by weight of ethyl acetate, and 10.0% by weight of water. The resulting mixture was charged at a charge flow rate of 100 g/hr into a reactor V and raised in temperature up to 70° C. The reactor V herein was a jacketed glass reactor having an inner diameter of 20 mm and a length of 300 mm and packed with 50 ml of a strong acid ion exchange resin.

A reactor outlet liquid had a composition including 3.2% by weight of ethanol, 32.4% by weight of ethyl acetate, 7.0% by weight of water, and 57.4% by weight of acetic acid. The reactor outlet liquid had an ethyl acetate to ethanol weight ratio of 10.1:1.0 which is about 11 times the ethyl acetate to ethanol weight ratio of the reactor inlet liquid of 0.91:1.0. This indicates that the reactor outlet liquid contributes to replenishment of ethyl acetate when charged typically into the absorber, the acetaldehyde product column, and/or the acetic acid recovery column.

### Example 7

A crude reaction liquid obtained by the method according to Example 1 was purified by the flow illustrated in FIG. 7.

A first distillation column (acetaldehyde product column) E herein included a glass distillation column including 30 theoretical trays, having a diameter of 50 mm, and equipped with a vacuum jacket. Onto the twentieth theoretical tray from the column top of the first distillation column E, the crude reaction liquid from the acetic acid hydrogenation was charged via the line 16, and subjected to distillation at normal atmospheric pressure at a reflux ratio of 3. An overhead vapor had a temperature of 21° C., was cooled down to 10° C., and drawn out via the line 18 to give acetaldehyde product at 120 g/hr. A bottom liquid had a temperature of 79° C. and was continuously drawn out via the line 19 at a rate of 1,547 g/hr so as to keep the liquid level constant. The bottom liquid included 2.1% by weight of acetone, 8.7% by weight of ethanol, 47.5% by weight of ethyl acetate, 11.0% by weight of water, and 30.8% by weight of acetic acid.

A second distillation column (acetic acid recovery column) F herein included a metallic distillation column including 30 theoretical trays and having a diameter of 100 mm. The bottom liquid was charged onto the twentieth theoretical tray of the second distillation column, and, in addition, an upper-phase liquid at 1,500 g/hr was charged via the line 23, and these were subjected to distillation at 190 kPa gauge pressure. The upper-phase liquid had been separated from the distillate of the second distillation column (acetic acid recovery column) F with the decanter S. An overhead vapor had a temperature of 103° C., was condensed into a distillate and cooled down to 20° C. with the condenser M-7, and was separated into an upper-phase liquid and a lower-phase liquid with the decanter S. Of the upper-phase liquid, 1,500 g/hr were refluxed to the second distillation column (acetic acid recovery column) F as described above, and 1,000 g/hr were recycled as the absorbing liquid to the acetic acid hydrogenation process.

A bottom liquid had a temperature of 157° C. and was continuously drawn out via the line 24 at a rate of 477 g/hr so as to keep the liquid level constant. The bottom liquid included 0.1% by weight of water and 99.9% by weight of acetic acid. The decanter lower-phase liquid was obtained at 79 g/hr and included 3.1% by weight of acetone, 13.8% by weight of ethanol, 13.0% by weight of ethyl acetate, and 70.1% by weight of water.

The lower-phase liquid was charged onto the tenth theoretical tray from the column top of a third distillation column (low-boiling component removal column) G, and subjected to distillation at normal atmospheric pressure at a reflux ratio of 210. The third distillation column G herein included a glass distillation column including 30 theoretical trays, having a diameter of 40 mm, and equipped with a vacuum jacket. An overhead vapor had a temperature of 59° C. and gave a distillate at 3 g/hr. The distillate included 79.2% by weight of acetone, 3.6% by weight of ethanol, 15.0% by weight of ethyl acetate, and 2.2% by weight of water. A bottom liquid had a temperature of 73° C. and was continuously drawn out via the line 28 at a rate of 76 g/hr so as to keep the liquid level constant. The bottom liquid included 14.2% by weight of ethanol, 12.9% by weight of ethyl acetate, and 72.9% by weight of water.

The bottom liquid was charged onto the fifth theoretical tray from the column top of a fourth distillation column (ethanol/ethyl acetate recovery column) H, and subjected to distillation at 40 kPa (absolute pressure), at a reflux ratio of 1.1. The fourth distillation column H herein included a glass distillation column including 10 theoretical trays, having a diameter of 40 mm, and equipped with a vacuum jacket. An overhead vapor had a temperature of 49° C. and gave a distillate at 23 g/hr. The distillate included 47.1% by weight of ethanol, 42.9% by weight of ethyl acetate, and 10.0% by weight of water. A bottom liquid had a temperature of 78° C. and was continuously drawn out via the line 31 at a rate of 53 g/hr so as to keep the liquid level constant. The bottom liquid included 0.1% by weight of ethanol and 99.9% by weight of water.

Acetic acid (245 parts by weight) was added to the distillate (100 parts by weight) from the ethanol/ethyl acetate recovery column, where the distillate included 47.1% by weight of ethanol, 42.9% by weight of ethyl acetate, and 10.0% by weight of water and had an ethanol to ethyl acetate weight ratio of 52:48. The resulting mixture was charged at a charge flow rate of 100 g/hr via the line 37 into a reactor V and raised in temperature up to 70° C. The reactor V herein was a jacketed glass reactor having an inner diameter of 20 mm and a length of 300 mm and packed with 50 ml of a strong acid ion exchange resin.

A reactor outlet liquid (line 38) had a composition including 3.2% by weight of ethanol, 32.4% by weight of ethyl acetate, 7.0% by weight of water, and 57.4% by weight of acetic acid and had an ethanol to ethyl acetate weight ratio of 9:91.

The ethanol to ethyl acetate weight ratio of 52:48 in the distillate of the ethanol/ethyl acetate recovery column is ethanol excess as compared with the ethanol to ethyl acetate weight ratio of 31:69 in the azeotropic composition of ethanol and ethyl acetate. This requires a complicated process to separate ethyl acetate from the distillate requires.

In contrast, the ethanol to ethyl acetate weight ratio of 9:91 in the reactor outlet liquid is ethyl acetate excess as compared with the azeotropic composition of ethanol and ethyl acetate. Because of this, ethyl acetate is easily separated from the reactor outlet liquid. Specifically, product ethyl acetate is obtained by subjecting the reactor outlet liquid to the ethyl acetate purification process X to separate and remove unreacted ethanol, water, and acetic acid from the reactor outlet liquid by a common procedure such as distillation and/or extraction.

### Example 8

An acetic acid hydrogenation was performed using the equipment illustrated in FIG. 9.

A gas from the top of an after-mentioned absorber (scrubber) C-1, passing sequentially through the line 12 and the line 32 at a rate of 1,073 NL/hr, was compressed with the compressor I-2 and recycled via the line 2. Hydrogen was fed at 94 NL/hr from the hydrogen cylinder P via the line 1, compressed with the compressor I-1, merged with the recycled gas, and charged via the line 3 into the evaporator A so that the evaporator A inlet pressure become constant at 1.7 MPa (gauge pressure). The equipment also included the buffer tanks J-1, J-2, and J-3.

Acetic acid was fed at a rate of 428 g/hr from the acetic acid tank K-1 via the line 4, and raised in temperature up to 300° C. in the evaporator (evaporator equipped with an electric heater) A together with the hydrogen fed via the line 3, to give a gaseous mixture of hydrogen and acetic acid. The gaseous mixture was charged into a reactor (reactor equipped with an electric heater) B. The reactor B herein had an outer diameter of 43.0 mm and was packed with 92 ml of a catalyst. The catalyst included 40 parts by weight of palladium (Pd) metal supported on 100 parts by weight of Fe₂O₃. The evaporator A and the reactor B each had an internal pressure of 1.7 MPa (gauge pressure). The reaction was performed at a temperature of 300° C. The equipment also included the pump N-1.

A reaction gas flown out from the reactor B via the line 6 was cooled down to 30° C. with the condenser (cooler) M-11 and charged via the line 7 into a lower portion of the absorber (scrubber) C-1. The absorber C-1 was packed with 6-mm diameter porcelain Raschig rings up to a height of 1 m and had an outer diameter of 48.6. The absorber (scrubber) C-1 had an internal pressure of 1.7 MPa (gauge pressure). The equipment also included the pump N-3 and the cooler M-4.

An absorbing liquid at 30° C. was charged at a rate of 63 g/hr via the line 33 into an upper portion of the absorber (scrubber) C-1. The absorbing liquid was a liquid having a composition corresponding to the upper-phase liquid in the line 48 as separated from the distillate of the acetic acid recovery column F with the decanter S in FIG. 11. The absorbing liquid included 0.9% by weight of acetone, 13.1% by weight of ethanol, 79.5% by weight of ethyl isobutyrate, and 6.5% by weight of water. The absorbing liquid was fed from the absorbing liquid tank K-9 using the pump N-16 via the line 34 through the cooler M-12.

A bottom liquid of the absorber (scrubber) C-1 was drawn out via the line 8 to the gas-liquid separator U at normal atmospheric pressure so as to keep the liquid level at the bottom of the absorber (scrubber) C-1 constant, and dissolved gases were stripped from the liquid. The stripped gases were separated and removed via the line 10. Part of the residual liquid after the gas stripping was charged (recycled) at a temperature of 30° C. and a rate of 3 L/hr via the line 9 into an intermediate portion of the absorber (scrubber) C-1.

The remainder of the liquid after the gas stripping was drawn out as a crude reaction liquid via the line 14 and stored in the crude reaction liquid tank K-2. The crude reaction liquid had a composition including 25.2% by weight of acetaldehyde, 0.4% by weight of acetone, 6.3% by weight of ethanol, 9.9% by weight of ethyl isobutyrate, 14.2% by weight of water, and 44.0% by weight of acetic acid. The crude reaction liquid was produced in an amount of 497 g/hr.

No gas purging from the column top gas line 12 of the absorber (scrubber) C-1 via the line 13 to the vent Q-1 was performed. However, a gas in the line 32 to be recycled to the evaporator A had a stable composition including 3.2% by mole of carbon dioxide, 1.1% by mole of methane, 1.2% by mole of ethane and ethylene, 0.7% by mole of propane and propylene, 0.2% by mole of acetaldehyde, and 93.6% by mole of hydrogen.

The crude reaction liquid obtained in the above manner was purified according to the flow illustrated in FIG. 11.

A first distillation column (acetaldehyde product column) E herein included a glass distillation column including 30 theoretical trays, having a diameter of 50 mm, and equipped with a vacuum jacket. Onto the twentieth theoretical tray from the column top of the first distillation column E, a liquid mixture of the crude reaction liquid from the acetic acid hydrogenation and a bottom liquid from an after-mentioned fifth distillation column (ethyl acetate separation column) was charged via the line 16 at a rate of 539 g/hr, and subjected to distillation at normal atmospheric pressure at a reflux ratio of 0.7. The liquid mixture included 23.3% by weight of acetaldehyde, 0.3% by weight of acetone, 6.5% by weight of ethanol, 9.8% by weight of ethyl isobutyrate, 14.2% by weight of water, and 45.9% by weight of acetic acid. An overhead vapor had a temperature of 21° C., was cooled down to 10° C., and drawn out via the line 18 to give acetaldehyde product at a rate of 130 g/hr. A bottom liquid had a temperature of 105° C. and was continuously drawn out via the line 19 at a rate of 409 g/hr so as to keep the liquid level constant. The bottom liquid included 0.4% by weight of acetone, 8.5% by weight of ethanol, 13.0% by weight of ethyl isobutyrate, 18.7% by weight of water, and 59.4% by weight of acetic acid.

The second distillation column (acetic acid recovery column) F herein included a metallic distillation column including 30 theoretical trays and having a diameter of 100 mm. The bottom liquid was charged onto the twentieth theoretical tray of the second distillation column F, and, in addition, an upper-phase liquid was charged at a rate of 563 g/hr via the line 23 into the second distillation column F, and distillation was performed at 190 kPa gauge pressure, where the upper-phase liquid had been separated from the distillate of the second distillation column (acetic acid recovery column) F with the decanter S. An overhead vapor had a temperature of 109° C., was condensed into a distillate and cooled down to 20° C. with the condenser M-7, and was separated into the upper-phase liquid and a lower-phase liquid with the decanter S. Of the upper-phase liquid, 563 g/hr were refluxed to the second distillation column (acetic acid recovery column) F as described above, and 63 g/hr were recycled as the absorbing liquid to the acetic acid hydrogenation process.

A bottom liquid had a temperature of 153° C. and was continuously drawn out via the line 24 at a rate of 256 g/hr so as to keep the liquid level constant. The bottom liquid included 5.6% by weight of ethyl isobutyrate and 94.4% by weight of acetic acid. The decanter lower-phase liquid was obtained at a rate of 105 g/hr and included 1.2% by weight of acetone, 25.5% by weight of ethanol, 3.4% by weight of ethyl isobutyrate, and 69.9% by weight of water.

The lower-phase liquid was charged onto the fifth theoretical tray from the column top of a third distillation column (low-boiling component removal column) G and subjected to distillation at normal atmospheric pressure at a reflux ratio of 25. The third distillation column herein included a glass distillation column including 30 theoretical trays, having a diameter of 40 mm, and equipped with a vacuum jacket. An overhead vapor had a temperature of 49° C. and gave a distillate at 2 g/hr. The distillate included 15.6% by weight of acetaldehyde, 69.4% by weight of acetone, 10.0% by weight of ethanol, 2.1% by weight of ethyl isobutyrate, and 2.9% by weight of water. A bottom liquid had a temperature of 85° C. and was continuously drawn out via the line 28 at a rate of 103 g/hr so as to keep the liquid level constant. The bottom liquid included 25.7% by weight of ethanol, 3.4% by weight of ethyl isobutyrate, and 70.9% by weight of water.

A fourth distillation column (ethanol recovery column) H herein included a glass distillation column including 20 theoretical trays, having a diameter of 40 mm, and equipped with a vacuum jacket. The bottom liquid was charged onto the fifteenth theoretical tray from the column top of the fourth distillation column H, and subjected to distillation at normal atmospheric pressure at a reflux ratio of 1.6. An overhead vapor had a temperature of 78° C. and gave a distillate at 33 g/hr. The distillate included 81.2% by weight of ethanol, 10.8% by weight of ethyl isobutyrate, and 8.0% by weight of water. A bottom liquid had a temperature of 102° C. and was continuously drawn out via the line 31 at a rate of 70 g/hr so as to keep the liquid level constant. The bottom liquid included 0.1% by weight of ethanol and 99.9% by weight of water.

Acetic acid (73 parts by weight) was added to the distillate (100 parts by weight) from the ethanol recovery column, where the distillate included 81.2% by weight of ethanol, 10.8% by weight of ethyl isobutyrate, and 8.0% by weight of water. The resulting mixture was charged at a charge flow rate of 100 g/hr via the line 49 into a reactor V and raised in temperature up to 70° C., followed by esterification. The reactor V herein was a jacketed glass reactor having an inner diameter of 20 mm and a length of 300 mm and packed with 50 ml of a strong acid ion exchange resin.

A reactor outlet liquid (line 38) had a composition including 10.3% by weight of ethanol, 40.3% by weight of ethyl acetate, 4.2% by weight of ethyl isobutyrate, 11.3% by weight of water, and 33.9% by weight of acetic acid.

The esterification reaction liquid was charged onto the tenth theoretical tray from the column top of a fifth distillation column (ethyl acetate separation column), and subjected to distillation at normal atmospheric pressure at a reflux ratio of 2.0. The fifth distillation column herein included a glass distillation column including 30 theoretical trays, having a diameter of 40 mm, and equipped with a vacuum jacket. An overhead vapor had a temperature of 70° C. and gave a distillate at 43 g/hr. The distillate included 11.8% by weight of ethanol, 79.4% by weight of ethyl acetate, 8.8% by weight of water. A bottom liquid had a temperature of 103° C. and was continuously drawn out via the line 47 at a rate of 41 g/hr so as to keep the liquid level constant. The bottom liquid included 8.8% by weight of ethanol, 8.5% by weight of ethyl isobutyrate, 14.0% by weight of water, and 68.7% by weight of acetic acid.

### REFERENCE SIGNS LIST

A evaporator
B reactor
C absorber
C-1 scrubber
D stripper
E first distillation column (acetaldehyde product column)
F second distillation column (acetic acid recovery column)
G third distillation column
H fourth distillation column
I-1 to I-2 compressor
J-1 to J-3 buffer tank
K-1 acetic acid tank
K-2 crude reaction liquid tank
K-3 acetaldehyde product tank
K-4 recovered acetic acid tank
K-5 ethyl acetate tank
K-6 absorbing liquid tank
K-7 low-boiling component tank
K-8 recovered ethanol/ethyl acetate tank
K-9 absorbing liquid tank
K-10 acetaldehyde product tank
K-11 esterification reaction liquid tank
K-12 ethyl acetate tank
L-1 to L-2 heater
M-1 to M-13 condenser (cooler)
N-1 to N-25 pump (delivery pump)
O-1 to O-4 reboiler
O-5 heater
O-6 reboiler
P hydrogen installation (hydrogen cylinder)
Q-1 to Q-3 vent
R-1 to R-5 receiver (tank)
S decanter
T waste water facility
U gas-liquid separator
V esterification reactor
W acetic acid
X ethyl acetate purification process
Y fifth distillation column (ethyl acetate separation column)
1 to 50 line

### INDUSTRIAL APPLICABILITY

The present invention is applicable to industrial production of acetaldehyde via acetic acid hydrogenation.

1. A method for producing acetaldehyde, the method comprising the steps of:
a) hydrogenating acetic acid to give a reaction fluid;
a′) charging the reaction fluid into an absorber, absorbing condensed components from the reaction fluid with an absorbing liquid, and dissolving non-condensable gases from the reaction fluid into the absorbing liquid; and
a″) reducing a pressure of a bottom liquid of the absorber to strip the dissolved non-condensable gases from the absorbing liquid, and recycling the resulting liquid after the non-condensable gas stripping to the absorber.

2. The method according to claim 1 for producing acetaldehyde, the method further comprising:
separating acetaldehyde from bottom liquid of the absorber to leave an aqueous acetic acid solution; and
using part of the aqueous acetic acid solution as the absorbing liquid in the absorber.

3. The method according to claim 1 for producing acetaldehyde, the method further comprising:
separating unreacted acetic acid and water from each other via azeotropic distillation using an azeotropic-solvent-containing liquid; and
using part of the azeotropic-solvent-containing liquid as the absorbing liquid in the absorber.

4. The method according to claim 1 for producing acetaldehyde,
wherein a solvent containing 10% by weight or more of an azeotropic solvent is used as the absorbing liquid in the absorber.

5. A method for producing acetaldehyde, the method comprising:
hydrogenating acetic acid to give a crude reaction liquid;
subjecting the crude reaction liquid to distillation in a distillation column; and
recovering acetaldehyde in a liquid phase from a tray disposed between a crude reaction liquid feed tray and a column top of the distillation column.

6. A method for producing acetaldehyde, the method comprising the steps of:
a) hydrogenating acetic acid to give a crude reaction liquid;
b) separating acetaldehyde from the crude reaction liquid via distillation in a first distillation column;
c) separating unreacted acetic acid from the resulting liquid after the acetaldehyde separation via distillation in a second distillation column;
d-1) separating a low-boiling component from the resulting liquid after the unreacted acetic acid separation via distillation in a third distillation column, the low-boiling component having a lower boiling point as compared with ethyl acetate; and
e-1) separating water and a mixture of ethanol and ethyl acetate from the resulting liquid after the low-boiling component separation via distillation in a fourth distillation column, or
the method comprising:
the step a);
the step b);
the step c);
d-2) the step of separating water from the resulting liquid after the unreacted acetic acid separation via distillation in a third distillation column; and
e-2) the step of separating a low-boiling component and a mixture of ethanol and ethyl acetate from the resulting liquid after the water separation via distillation in a fourth distillation column, the low-boiling component having a lower boiling point as compared with ethyl acetate.

7. The method according to claim 6 for producing acetaldehyde, the method further comprising:
controlling pressures upon operation so that a temperature of an overhead vapor of the second distillation column is higher than a bottom temperature of at least one distillation column selected from the group consisting of the first distillation column, the third distillation column, and the fourth distillation column; and
using the overhead vapor of the second distillation column as a heat source for heating at least one distillation column selected from the group consisting of the first distillation column, the third distillation column, and the fourth distillation column.

8. A method for producing acetaldehyde, the method comprising the steps of:
a) hydrogenating acetic acid to give a crude reaction liquid;
b) separating acetaldehyde from the crude reaction liquid via distillation in a first distillation column;
c′) separating unreacted acetic acid from the resulting liquid after the acetaldehyde separation via distillation using ethyl acetate as an azeotropic solvent in a second distillation column;
d-1) separating a low-boiling component from the resulting liquid after the unreacted acetic acid separation via distillation in a third distillation column, the low-boiling component having a lower boiling point as compared with ethyl acetate;
e-1) separating water and a mixture of ethanol and ethyl acetate from the resulting liquid after the low-boiling component separation via distillation in a fourth distillation column;
f) adding acetic acid to part or a whole of the mixture of ethanol and ethyl acetate and esterifying the ethanol into ethyl acetate in the presence of an acid catalyst; and
g) recycling ethyl acetate as the azeotropic solvent, or
the method comprising:
the step a);
the step b);
the step c′);
d-2) the step of separating water from the resulting liquid after the unreacted acetic acid separation via distillation in a third distillation column;
e-2) the step of separating a low-boiling component and a mixture of ethanol and ethyl acetate from the resulting liquid after the water separation via distillation in a fourth distillation column, the low-boiling component having a lower boiling point as compared with ethyl acetate;
the step f); and
the step g).

9. A method for producing acetaldehyde and ethyl acetate, the method comprising the steps of:
a) hydrogenating acetic acid to give a crude reaction liquid;
b) separating acetaldehyde from the crude reaction liquid via distillation in a first distillation column;
c′) separating unreacted acetic acid from the resulting liquid after the acetaldehyde separation via distillation using ethyl acetate as an azeotropic solvent in a second distillation column;
d-1) separating a low-boiling component from the resulting liquid after the unreacted acetic acid separation via distillation in a third distillation column, the low-boiling component having a lower boiling point as compared with ethyl acetate;
e-1) separating water and a mixture of ethanol and ethyl acetate from the resulting liquid after the low-boiling component separation via distillation in a fourth distillation column;
f) adding acetic acid to part or a whole of the mixture of ethanol and ethyl acetate and esterifying the ethanol into ethyl acetate in the presence of an acid catalyst; and
h) recovering the ethyl acetate as a product, or
the method comprising:
the step a);
the step b);
the step c′);
d-2) the step of separating water from the resulting liquid after the unreacted acetic acid separation via distillation in a third distillation column;
e-2) the step of separating a low-boiling component and a mixture of ethanol and ethyl acetate from the resulting liquid after the water separation via distillation in a fourth distillation column, the low-boiling component having a lower boiling point as compared with ethyl acetate;
the step f); and
the step h).

10. A method for producing acetaldehyde and ethyl acetate, the method comprising the steps of:
a) hydrogenating acetic acid to give a crude reaction liquid;
b) separating acetaldehyde from the crude reaction liquid via distillation in a first distillation column;
c′) separating unreacted acetic acid from the resulting liquid after the acetaldehyde separation via distillation using an azeotropic solvent in a second distillation column;
d-1) separating a low-boiling component from the resulting liquid after the unreacted acetic acid separation via distillation in a third distillation column, the low-boiling component having a lower boiling point as compared with ethanol;
e-1) separating water and a mixture of ethanol and the azeotropic solvent from the resulting liquid after the low-boiling component separation via distillation in a fourth distillation column;
f) adding acetic acid to part or a whole of the mixture of ethanol and the azeotropic solvent and esterifying the ethanol into ethyl acetate in the presence of an acid catalyst to give an esterification reaction liquid; and
i) subjecting the esterification reaction liquid to distillation in a fifth distillation column to recover the ethyl acetate as an overhead product and to recover the azeotropic solvent as a bottom liquid, and recycling the recovered azeotropic solvent, or
the method comprising:
the step a);
the step b);
the step c′);
d-2) the step of separating water from the resulting liquid after the unreacted acetic acid separation via distillation in a third distillation column;
e-2) the step of separating a low-boiling component and a mixture of ethanol and the azeotropic solvent from the resulting liquid after the water separation via distillation in a fourth distillation column, the low-boiling component having a lower boiling point as compared with ethanol;
the step f); and
the step i).

11. The method according to claim 10 for producing acetaldehyde and ethyl acetate,
wherein the azeotropic solvent includes an ester having a boiling point of 100° C. to 118° C. at normal atmospheric pressure.

12. The method according to one of claims 10 and 11 for producing acetaldehyde and ethyl acetate, the method further comprising
controlling pressures upon operation so that a temperature of an overhead vapor of the second distillation column is higher than a bottom temperature of at least one distillation column selected from the group consisting of the first distillation column, the third distillation column, the fourth distillation column, and the fifth distillation column; and
using the overhead vapor of the second distillation column as a heat source for heating at least one distillation column selected from the group consisting of the first distillation column, the third distillation column, the fourth distillation column, and the fifth distillation column.
